(19) United States
(12) Patent Application Publication
(10) Pub. No.: US 2011/0105564 A1
(43) Pub. Date: May 5, 2011

(54) THIENYLPYRIDYLCARBOXAMIDES

(75) Inventors: Ralf Dunkel, Lyon (FR); Hans-Ludwig Elbe, Wuppertal (DE); Jörg Nico Greul, Leichlingen (DE); Herbert Gayer, Monheim (DE); Benoit Hartmann, Langenfeld (DE); Arnd Voerste, Koln (DE); Ulrike Wachendorff-Neumann, Neuwied (DE); Peter Dahmen, Neuss (DE)

(73) Assignee: Bayer CropScience AG, Monheim Am Rhein (DE)

(21) Appl. No.: 12/097,501

(22) PCT Filed: Dec. 5, 2006
(86) PCT No.: PCT/EP2006/011650

§ 371 Date: Nov. 17, 2008

(30) Foreign Application Priority Data
Dec. 17, 2005 (DE) 102005060468.4

(51) Int. Cl. A01N 43/40; C07D 401/14; C07F 5/02; C07D 333/36; A01P 3/00

(52) U.S. Cl. 514/333; 546/256; 546/13; 549/69

(57) ABSTRACT

Novel thienylpyridylcarboxamides of the formula (I)

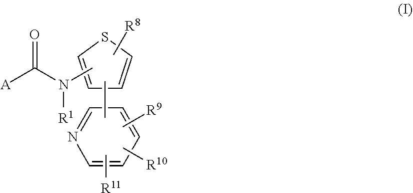

The present application is also directed to a plurality of processes for preparing these compounds and their use for controlling unwanted microorganisms, and also novel intermediates and their preparation.

The present invention relates to novel thienylpyridylcarboxamides, to a plurality of processes for their preparation and to their use for controlling harmful microorganisms in crop protection and in the protection of materials.

It is already known that certain thienylpyridylcarboxamides have fungicidal properties (cf., for example, WO 01/53259 and WO 01/49664). The activity of the substances described in these publications is good; however, it is sometimes unsatisfactory at low application rates.

This invention now provides novel thienylpyridylcarboxamides of the formula (I)

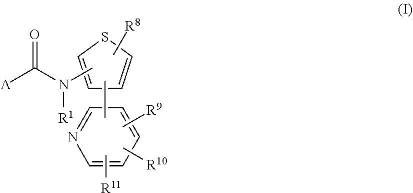

in which
R¹ represents hydrogen, C₁-C₈-alkyl, C₁-C₆-alkylsulphinyl, C₁-C₆-alkylsulphonyl, C₁-C₄-alkoxy-C₁-C₄-alkyl, C₃-C₈-cycloalkyl; C₁-C₆-haloalkyl, C₁-C₄-haloalkylthio, C₁-C₄-haloalkylsulphinyl, C₁-C₄-haloalkylsulphonyl, halo-C₁-C₄-alkoxy-C₁-C₄-alkyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₁-C₃-alkoxy)carbonyl-C₁-C₃-alkyl having in each case 1 to 13 fluorine, chlorine and/or bromine atoms; (C₁-C₈-alkyl)carbonyl, (C₁-C₈alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl, (C₃-C₈-cycloalkyl)carbonyl; (C₁-C₆-haloalkyl)carbonyl, (C₁-C₆-haloalkoxy)carbonyl, (C₁-C₆-haloalkylthio)carbonyl, (halo-C₁-C₄-alkoxy-C₁-C₄-alkyl)carbonyl, (C₃-C₆-haloalkenyloxy)carbonyl, (C₃-C₆-haloalkynyloxy)carbonyl, (C₃-C₈-halocycloalkyl)carbonyl having in each case 1 to 9 fluorine, chlorine and/or bromine atoms; or —CH₂—C≡C—R^{1-A}, —CH₂—CH═CH—R^{1-A}, —CH═C═CH—R^{1-A}, —C(═O)C(═O)R², —CONR³R⁴ or —CH₂NR⁵R⁶,
R^{1-A} represents hydrogen, C₁-C₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)carbonyl or cyano,
R² represents hydrogen, C₁-C₈-alkyl, C₁-C₈-alkoxy, C₁-C₄-alkoxy-C₁-C₄-alkyl, C₃-C₈-cycloalkyl; C₁-C₆-haloalkyl, C₁-C₆-haloalkoxy, halo-C₁-C₄-alkoxy-C₁-C₄-alkyl, C₃-C₈-halocycloalkyl having in each case 1 to 9 fluorine, chlorine and/or bromine atoms,
R³ and R⁴ independently of one another each represent hydrogen, C₁-C₈-alkyl, C₁-C₄-alkoxy-C₁-C₄-alkyl, C₃-C₈-cycloalkyl; C₁-C₈-haloalkyl, halo-C₁-C₄-alkoxy-C₁-C₄-alkyl, C₃-C₈-halocycloalkyl having in each case 1 to 9 fluorine, chlorine and/or bromine atoms,
R³ and R⁴ furthermore together with the nitrogen atom to which they are attached form a saturated heterocycle which is mono- or polysubstituted by identical or different substituents from the group consisting of halogen and C₁-C₄-alkyl and which has 5 to 8 ring atoms, where the heterocycle may contain 1 or 2 further non-adjacent heteroatoms from the group consisting of oxygen, sulphur and NR⁷,
R⁵ and R⁶ independently of one another each represent hydrogen, C₁-C₈-alkyl, C₃-C₈-cycloalkyl; C₁-C₈-haloalkyl, C₃-C₈-halocycloalkyl having in each case 1 to 9 fluorine, chlorine and/or bromine atoms,
R⁵ and R⁶ furthermore together with the nitrogen atom to which they are attached form a saturated heterocycle which is mono- or polysubstituted by identical or different substituents from the group consisting of halogen and C₁-C₄-alkyl and which has 5 to 8 ring atoms, where the heterocycle may contain 1 or 2 further non-adjacent heteroatoms from the group consisting of oxygen, sulphur and NR⁷,
R⁷ represents hydrogen or C₁-C₆-alkyl,
R⁸ represents hydrogen, fluorine, chlorine, methyl, isopropyl, methylthio or trifluoromethyl,
R⁹, R¹⁰ and R¹¹ independently of one another represent hydrogen, halogen, cyano, nitro, C₁-C₆-alkyl, C₂-C₆-alkenyl, C₂-C₆-alkynyl, C₁-C₄-alkoxy, C₁-C₄-alkylthio, C₁-C₄-alkylsulphinyl, C₁-C₄-alkylsulphonyl, C₃-C₆-cycloalkyl; represent C₁-C₄-haloalkyl, C₁-C₄-haloalkoxy, C₁-C₄-haloalkylthio, C₁-C₄-haloalkylsulphinyl, C₁-C₄-haloalkylsulphonyl having in each case 1 to 5 halogen atoms; represent —SO₂NR¹²R¹³, —C(═X)R¹⁴, —Si(R¹⁵)₃, C₂-C₄-alkenylene-Si(R¹⁵)₃, C₂-C₄-alkenylene-Si(R¹⁵)₃, —NR¹²R¹³,
R¹² represents hydrogen, C₁-C₄-alkyl or —C(═X)R¹⁴,
R¹³ represents hydrogen, C₁-C₄-alkyl or —C(═X)R¹⁴,
R¹² and R¹³ furthermore together with the nitrogen atom to which they are attached form a saturated heterocycle which is optionally mono- or polysubstituted by identical or different substituents from the group consisting of halogen and C₁-C₄-alkyl and which has 5 to 8 ring atoms, where the heterocycle may contain 1 or 2 further non-adjacent heteroatoms from the group consisting of oxygen, sulphur and NR⁷,
X represents O (oxygen) or S (sulphur),
R¹⁴ represents hydrogen, C₁-C₄alkyl, C₁-C₄-alkoxy or —NR¹⁶R¹⁷,
R¹⁵ represents hydrogen, C₁-C₈-alkyl, C₁-C₈-alkoxy, C₁-C₄-alkoxy-C₁-C₄-alkyl, C₁-C₄-alkylthio-C₁-C₄-alkyl or C₁-C₆-haloalkyl, where the three radicals R¹⁵ may each be identical or different,
R¹⁶ represents hydrogen or C₁-C₄-alkyl,
R¹⁷ represents hydrogen or C₁-C₄-alkyl,
R¹⁶ and R¹⁷ furthermore together with the nitrogen atom to which they are attached form a saturated heterocycle which is optionally mono- or polysubstituted by identical or different substituents from the group consisting of halogen and C₁-C₄-alkyl and which has 5 to 8 ring atoms, where the heterocycle may contain 1 or 2 further non-adjacent heteroatoms from the group consisting of oxygen, sulphur and NR⁷,
A represents one of the radicals A1 to A18 below

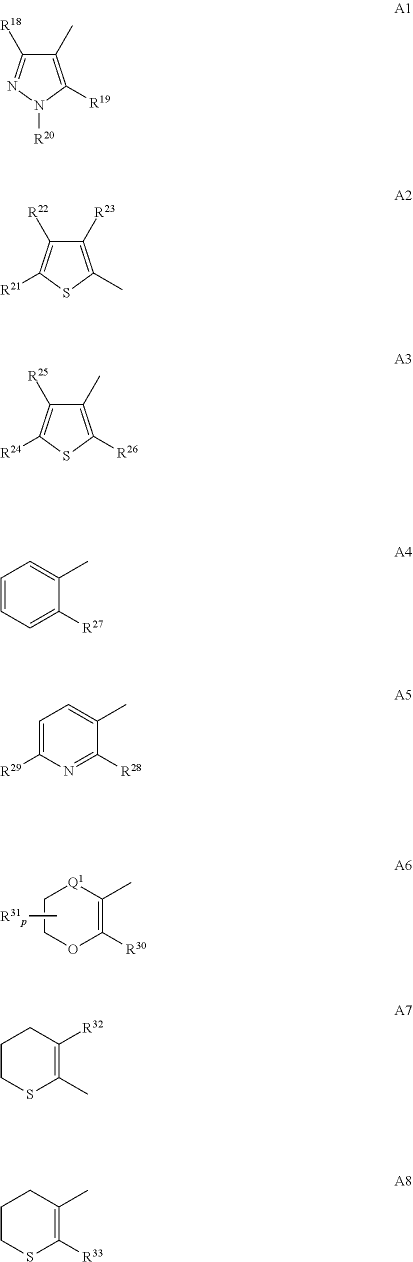

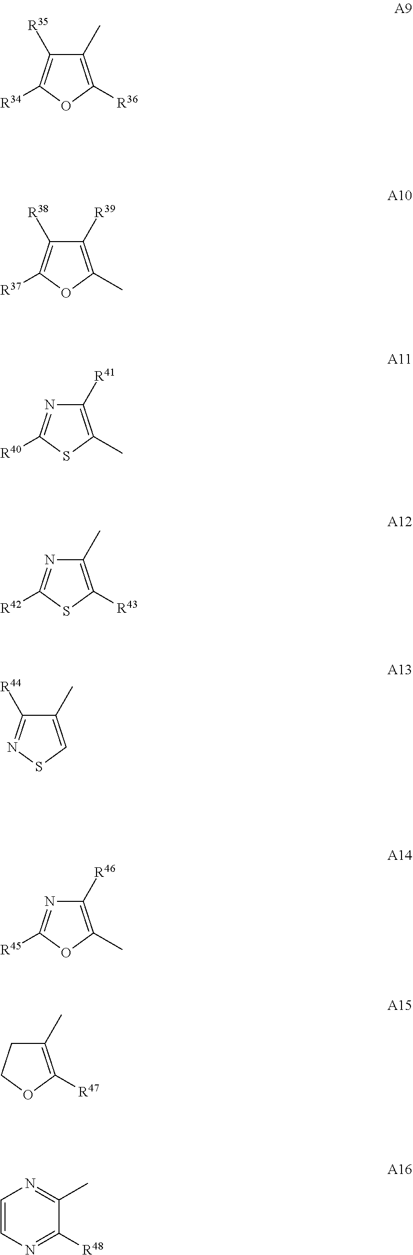

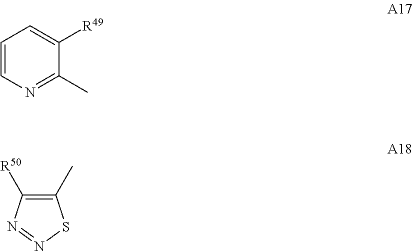

R¹⁸ represents hydrogen, cyano, halogen, nitro, C₁-C₄-alkyl, C₁-C₄-alkoxy, C₁-C₄-alkylthio, C₃-C₆-cycloalkyl, C₁-C₄-haloalkyl, C₁-C₄-haloalkoxy or C₁-C₄-haloalkylthio having in each case 1 to 5 halogen atoms, aminocarbonyl or aminocarbonyl-C₁-C₄-alkyl,
R¹⁹ represents hydrogen, halogen, cyano, C₁-C₄-alkoxy or C₁-C₄-alkylthio,
R²⁰ represents hydrogen, C₁-C₄-alkyl, hydroxy-C₁-C₄-alkyl, C₂-C₆-alkenyl, C₃-C₆-cycloalkyl, C₁-C₄-alkylthio-C₁-C₄alkyl, C₁-C₄-alkoxy-C₁-C₄alkyl, C₁-C₄-haloalkyl, C₁-C₄-haloalkylthio-C₁-C₄-alkyl, C₁-C₄haloalkoxy-C₁-C₄alkyl having in each case 1 to 5 halogen atoms, or phenyl,
R²¹ and R²² independently of one another represent hydrogen, halogen, C₁-C₄-alkyl or C₁-C₄-haloalkyl having 1 to 5 halogen atoms,
R²³ represents halogen, cyano or C₁-C₄-alkyl, or C₁-C₄haloalkyl or C₁-C₄-haloalkoxy having in each case 1 to 5 halogen atoms,
R²⁴ and R²⁵ independently of one another represent hydrogen, halogen, C₁-C₄-alkyl or C₁-C₄-haloalkyl having 1 to 5 halogen atoms,
R²⁶ represents hydrogen, halogen, C₁-C₄-alkyl or C₁-C₄-haloalkyl having 1 to 5 halogen atoms,
R²⁷ represents hydrogen, halogen, hydroxyl, cyano, C₁-C₆-alkyl, C₁-C₄-haloalkyl, C₁-C₄-haloalkoxy or C₁-C₄-haloalkylthio having in each case 1 to 5 halogen atoms,
R²⁸ represents halogen, hydroxyl, cyano, C₁-C₄-alkyl, C₁-C₄-alkoxy, C₁-C₄-alkylthio, C₁-C₄-haloalkyl, C₁-C₄haloalkylthio or C₄-C₄haloalkoxy having in each case 1 to 5 halogen atoms,
R²⁹ represents hydrogen, halogen, cyano, C₁-C₄alkyl, C₁-C₄-alkoxy, C₁-C₄-alkylthio, C₁-C₄-haloalkyl, C₁-C₄-haloalkoxy having in each case 1 to 5 halogen atoms, C₁-C₄alkylsulphinyl or C₁-C₄-alkylsulphonyl,

R³⁰ represents C₁-C₄-alkyl or C₁-C₄-haloalkyl having 1 to 5 halogen atoms, R³¹ represents C₁-C₄-alkyl,
Q¹ represents S (sulphur), SO, SO₂ or CH₂,
p represents 0, 1 or 2, where R²² represents identical or different radicals if p represents 2,
R³² represents C₁-C₄-alkyl or C₁-C₄haloalkyl having 1 to 5 halogen atoms,
R³³ represents C₁-C₄-alkyl or C₁-C₄-haloalkyl having 1 to 5 halogen atoms,
R³⁴ and R³⁵ independently of one another represent hydrogen, halogen, amino, C₁-C₄-alkyl or C₁-C₄-haloalkyl having 1 to 5 halogen atoms,
R³⁶ represents hydrogen, halogen, C₁-C₄-alkyl or C₁-C₄-haloalkyl having 1 to 5 halogen atoms,
R³⁷ and R³⁸ independently of one another represent hydrogen, halogen, amino, nitro, C₁-C₄-alkyl or C₁-C₄-haloalkyl having 1 to 5 halogen atoms,
R³⁹ represents hydrogen, halogen, C₁-C₄-alkyl or C₁-C₄haloalkyl having 1 to 5 halogen atoms,
R⁴⁰ represents hydrogen, halogen, amino, C₁-C₄-alkylamino, di-(C₁-C₄-alkyl)amino, cyano, C₁-C₄-alkyl or C₁-C₄-haloalkyl having 1 to 5 halogen atoms,
R⁴¹ represents halogen, C₁-C₄-alkyl or C₁-C₄haloalkyl having 1 to 5 halogen atoms,
R⁴² represents hydrogen, halogen, amino, C₁-C₄-alkylamino, di-(C₁-C₄-alkyl)amino, cyano, C₁-C₄-alkyl or C₁-C₄-haloalkyl having 1 to 5 halogen atoms,
R⁴³ represents halogen, C₁-C₄-alkyl or C₁-C₄-haloalkyl having 1 to 5 halogen atoms,
R⁴⁴ represents halogen, C₁-C₄-alkyl or C₁-C₄-haloalkyl having 1 to 5 halogen atoms,
R⁴⁵ represents hydrogen or C₁-C₄-alkyl,
R⁴⁶ represents halogen or C₁-C₄-alkyl,
R⁴⁷ represents C₁-C₄-alkyl or C₁-C₄-haloalkyl having 1 to 5 halogen atoms,
R⁴⁸ represents hydrogen, halogen, C₁-C₄-alkyl or C₁-Cₐ-haloalkyl having 1 to 5 halogen atoms,
R⁴⁹ represents halogen, hydroxyl, C₁-C₄-alkyl, C₁-C₄-alkoxy, C₁-C₄-alkylthio, C₁-C₄-haloalkyl, C₁-C₄-haloalkylthio or C₁-C₄-haloalkoxy having in each case 1 to 5 halogen atoms,
R⁵⁰ represents C₁-C₄-alkyl.

Furthermore, it has been found that thienylpyridylcarboxamides of the formula (I) are obtained when
(a) carbonyl halides of the formula (II)

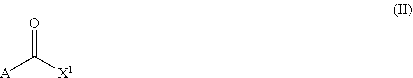

in which
A is as defined above,
X¹ represents halogen or hydroxyl,
are reacted with amines of the formula (III)

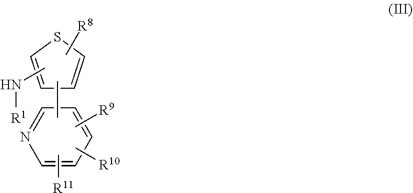

in which R¹, R⁸, R⁹, R¹⁰ and R¹¹ are as defined above,
if appropriate in the presence of a coupling agent, if appropriate in the presence of an acid binder and if appropriate in the presence of a diluent,

or
(b) thienylpyridylcarboxamides of the formula (I-a)

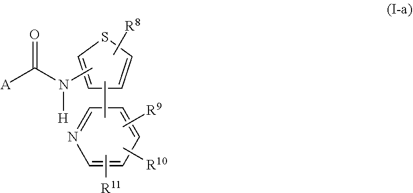

in which R⁸, R⁹, R¹⁰, R¹¹ and A are as defined above,
are reacted with halides of the formula (IV)

R^{1-B}-Hal (IV)
in which
R^{1-B} represents C₁-C₈-alkyl, C₁-C₆-alkylsulphinyl, C₁-C₆-alkylsulphonyl, C₁-C₄-alkoxy-C₁-C₄-alkyl, C₃-C₈-cycloalkyl; C₁-C₆-haloalkyl, C₁-C₄-haloalkylthio, C₁-C₄-haloalkylsulphinyl, C₁-C₄-haloalkylsulphonyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₁-C₃-alkoxy)carbonyl-C₁-C₃-alkyl having in each case 1 to 13 fluorine, chlorine and/or bromine atoms; (C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl, (C₃-C₈-cycloalkyl)carbonyl; (C₁-C₆-haloalkyl)carbonyl, (C₁-C₆-haloalkoxy)carbonyl, (C₁-C₆-haloalkylthio)carbonyl, (halo-C₁-C₄-alkoxy-C₁-C₄-alkyl)carbonyl, (C₃-C₆-haloalkenyloxy)carbonyl, (C₃-C₆-haloalkynyloxy)carbonyl, (C₃-C₈-halocycloalkyl)carbonyl having in each case 1 to 9 fluorine, chlorine and/or bromine atoms; or —CH₂—C≡C—R^{1-A}, —CH₂—CH═CH—R^{1-A}, —CH═C═CH—R^{1-A}, —C(═O)C(═O)R², —CONR³R⁴ or —CH₂NR⁵R⁶, R^{1-A}, R², R³, R⁴, R⁵ and R⁶ are as defined above,
Hal represents chlorine, bromine or iodine
in the presence of a base and in the presence of a diluent.

Finally, it has been found that the novel thienylpyridylcarboxamides of the formula (I) have very good microbicidal properties and can be used for controlling unwanted microorganisms both in crop protection and in the protection of materials.

If appropriate, the compounds according to the invention can be present as mixtures of different possible isomeric forms, in particular of stereoisomers, such as, for example, E and Z, threo and erythro, and also optical isomers, and, if appropriate, also of tautomers. What is claimed are both the E and the Z isomers, and also the threo and erythro, and the optical isomers, any mixtures of these isomers, and the possible tautomeric forms.

The formula (I) provides a general definition of the biphenylthiazolecarboxamides according to the invention. Preferred radical definitions of the formulae mentioned above and below are stated below. These definitions apply to the end products of the formula (I) and likewise to all intermediates.
preferably represents hydrogen, C₁-C₆-alkyl, C₁-C₄-alkylsulphinyl, C₁-C₄-alkylsulphonyl, C₁-C₃-alkoxy-C₁-C₃-alkyl, C₃-C₆-cycloalkyl; C₁-C₄-haloalkyl, C₁-C₄-haloalkylthio, C₁-C₄-haloalkylsulphinyl, C₁-C₄-haloalkylsulphonyl, halo-C₁-C₃-alkoxy-C₁-C₃-alkyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₁-C₃-alkoxy)carbonyl-C₁-C₃-alkyl having in each case 1 to 13 fluorine, chlorine and/or bromine atoms; (C₁-C₆-alkyl)carbonyl, (C₁-C₄-alkoxy)carbonyl, (C₁-C₄-alkylthio)carbonyl, (C₁-C₃-alkoxy-C₁-C₃-alkyl)carbonyl, (C₃-C₄-alkenyloxy)carbonyl, (C₃-C₄-alkynyloxy)carbonyl, (C₃-C₆-cycloalkyl)carbonyl; (C₁-C₄-haloalkyl)carbonyl, (C₁-C₄-haloalkoxy)carbonyl, (C₁-C₄-haloalkylthio)carbonyl, (halo-C₁-C₃-alkoxy-C₁-C₃-alkyl)carbonyl, (C₃-C₄-haloalkenyloxy)carbonyl, (C₃-C₄-haloalkynyloxy)carbonyl, (C₃-C₆-halocycloalkyl)carbonyl having in each case 1 to 9 fluorine, chlorine and/or bromine atoms; or —CH₂—C≡C—R^{1-A}, —CH₂—CH═CH—R^{1-A}, —CH═C═CH—R^{1-A}, —C(═O)C(═O)R², —CONR³R⁴ or —CH₂NR⁵R⁶.
R¹ particularly preferably represents hydrogen, methyl, ethyl, n- or isopropyl, n-, iso-, sec- or tert-butyl, pentyl or hexyl, methylsulphinyl, ethylsulphinyl, n- or isopropylsulphinyl, n-, iso-, sec- or tert-butylsulphinyl, methylsulphonyl, ethylsulphonyl, n- or isopropylsulphonyl, n-, iso-, sec- or tert-butylsulphonyl, methoxymethyl, methoxyethyl, ethoxymethyl, ethoxyethyl, cyclopropyl, cyclopentyl, cyclohexyl, trifluoromethyl, trichloromethyl, trifluoroethyl, difluoromethylthio, difluorochloromethylthio, trifluoromethylthio, trifluoromethylsulphinyl, trifluoromethylsulphonyl, trifluoromethoxymethyl; formyl, —CH₂—CHO, —(CH₂)₂—CHO, —CH₂—CO—CH₃, —CH₂—CO—CH₂CH₃, —CH₂—CO—CH(CH₃)₂, —(CH₂)₂—CO—CH₃, —(CH₂)₂—CO—CH₂CH₃, —(CH₂)—CO—CH(CH₃)₂, —CH₂—CO₂CH₃, —CH₂—CO₂CH₂CH₃, —CH₂—CO₂CH(CH₃)₂, —(CH₂)₂—CO₂CH₃, —(CH₂)₂—CO₂CH₂CH₃, —(CH₂)₂—CO₂CH(CH₃)₂, —CH₂—CO—CF₃, —CH₂—CO—CCl₃, —CH₂—CO—CH₂CF₃, —CH₂—CO—CH₂CCl₃, —(CH₂)₂—CO—CH₂CF₃, —(CH₂)₂—CO—CH₂CCl₃, —CH₂—CO₂CH₂CF₃, —CH₂—CO₂CF₂CF₃, —CH₂—CO₂CH₂CCl₃, —CH₂—CO₂CCl₂CCl₃, —CH₂)₂—CO₂CH₂CF₃, —(CH₂)₂—CO₂CF₂CF₃, —(CH₂)₂—CO₂CH₂CCl₃, —(CH₂)₂—CO₂CCl₂CCl₃; methylcarbonyl, ethylcarbonyl, n-propylcarbonyl, isopropylcarbonyl, tert-butylcarbonyl, methoxycarbonyl, ethoxycarbonyl, isopropoxycarbonyl, tert-butoxycarbonyl, methylthiocarbonyl, ethylthiocarbonyl, isopropylthiocarbonyl, tert-butylthiocarbonyl, methoxymethylcarbonyl, ethoxymethylcarbonyl, cyclopropylcarbonyl; trifluoromethylcarbonyl, trifluoromethoxycarbonyl, trifluoromethylthiocarbonyl, or —CH₂—C≡C—R^{1-A}, —CH₂—CH═CH—R^{1-A}, —CH═C═CH—R^{1-A}, —C(═O)C(═O)R², —CONR³R⁴ or —CH₂NR⁵R⁶.
R¹ very particularly preferably represents hydrogen, methyl, methoxymethyl, methoxymethylcarbonyl, ethoxymethylcarbonyl, formyl, —CH₂—CH═CH₂, —CH═C═CH₂, —CH₂—CHO, —(CH₂)₂—CHO, —CH₂—CO—CH₃, —CH₂—CO—CH₂CH₃, —CH₂—CO—CH(CH₃)₂, —C(═O)CHO, —C(═O)C(═O)CH₃, —C(═O)C(═O)CH₂OCH₃, —C(═O)CO₂CH₃, —C(═O)CO₂CH₂CH₃.
R^{1-A} preferably represents hydrogen, C₁-C₄-alkyl, C₁-C₄-haloalkyl, C₂-C₄-alkenyl, C₂-C₄-alkynyl, C₃-C₆-cycloalkyl, (C₁-C₄-alkoxy)carbonyl, or cyano.
R^{1-A} particularly preferably represents hydrogen, methyl or ethyl.
R² preferably represents hydrogen, C₁-C₆-alkyl, C₁-C₄-alkoxy, C₁-C₃-alkoxy-C₁-C₃-alkyl, C₃-C₆-cycloalkyl; C₁-C₄-haloalkyl, C₁-C₄-haloalkoxy, halo-C₁-C₃-alkoxy-C₁-C₃-alkyl, C₃-C₆-halocycloalkyl having in each case 1 to 9 fluorine, chlorine and/or bromine atoms.
R² particularly preferably represents hydrogen, methyl, ethyl, n- or isopropyl, tert-butyl, methoxy, ethoxy, n- or iso-propoxy, tert-butoxy, methoxymethyl, cyclopropyl; trifluoromethyl, trifluoromethoxy.
R³ and R⁴ independently of one another preferably represent hydrogen, C₁-C₆-alkyl, C₁-C₃-alkoxy-C₁-C₃-alkyl, C₃-C₆-cycloalkyl; C₁-C₄-haloalkyl, halo-C₁-C₃-alkoxy-C₁-C₃-alkyl, C₃-C₆-halocycloalkyl having in each case 1 to 9 fluorine, chlorine and/or bromine atoms.
R³ and R⁴ furthermore together with the nitrogen atom to which they are attached preferably form a saturated heterocycle which is optionally mono- to tetrasubstituted by identical or different substituents from the group consisting of halogen and C₁-C₄-alkyl and which has 5 or 6 ring atoms, where the heterocycle may contain 1 or 2 further non-adjacent heteroatoms from the group consisting of oxygen, sulphur and NR⁷.
R³ and R⁴ independently of one another particularly preferably represent hydrogen, methyl, ethyl, n- or isopropyl, n-, iso-, sec- or tert-butyl, methoxymethyl, methoxyethyl, ethoxymethyl, ethoxyethyl, cyclopropyl, cyclopentyl, cyclohexyl; trifluoromethyl, trichloromethyl, trifluoroethyl, trifluoromethoxymethyl.
R³ and R⁴ furthermore together with the nitrogen atom to which they are attached particularly preferably form a saturated heterocycle from the group consisting of morpholine, thiomorpholine and piperazine which is optionally mono- to tetrasubstituted by identical or different substituents from the group consisting of fluorine, chlorine, bromine and methyl, where the piperazine may be substituted on the second nitrogen atom by R⁷.
R⁵ and R⁶ independently of one another preferably represent hydrogen, C₁-C₆-alkyl, C₃-C₆-cycloalkyl; C₁-C₄-haloalkyl, C₃-C₆-halocycloalkyl having in each case 1 to 9 fluorine, chlorine and/or bromine atoms.
R⁵ and R⁶ furthermore together with the nitrogen atom to which they are attached preferably form a saturated heterocycle which is optionally mono- or polysubstituted by identical or different substituents from the group consisting of halogen and C₁-C₄-alkyl and which has 5 or 6 ring atoms, where the heterocycle may contain 1 or 2 further non-adjacent heteroatoms from the group consisting of oxygen, sulphur and NR⁷.
R⁵ and R⁶ independently of one another particularly preferably represent hydrogen, methyl, ethyl, n- or isopropyl, n-, iso-, sec- or tert-butyl, methoxymethyl, methoxyethyl, ethoxymethyl, ethoxyethyl, cyclopropyl, cyclopentyl, cyclohexyl; trifluoromethyl, trichloromethyl, trifluoroethyl, trifluoromethoxymethyl.
R⁵ and R⁶ furthermore together with the nitrogen atom to which they are attached particularly preferably form a saturated heterocycle from the group consisting of morpholine, thiomorpholine and piperazine which is optionally mono- to tetrasubstituted by identical or different substituents from the group consisting of fluorine, chlorine, bromine and methyl, where the piperazine may be substituted on the second nitrogen atom by R⁷.

R⁷ preferably represents hydrogen or C₁-C₄-alkyl.
R⁷ particularly preferably represents hydrogen, methyl, ethyl, n- or isopropyl, n-, iso-, sec- or tert-butyl.
R⁸ preferably represents hydrogen, chlorine or methyl.
R⁸ particularly preferably represents hydrogen.
R⁸ furthermore particularly preferably represents chlorine.
R⁸ furthermore particularly preferably represents methyl.
R⁹, R¹⁰ and R¹¹ independently of one another preferably represent hydrogen, fluorine, chlorine, bromine, iodine, cyano, nitro, C₁-C₆-alkyl, C₂-C₄-alkenyl, C₂-C₄-alkynyl, C₁-C₄-alkoxy, C₁-C₄-alkylthio, C₁-C₄-alkylsulphinyl, C₁-C₄-alkylsulphonyl, C₃-C₆-cycloalkyl; represent C₁-C₄-haloalkyl, C₁-C₄-haloalkoxy, C₁-C₄-haloalkylthio, C₁-C₄-haloalkylsulphinyl, C₁-C₄-haloalkylsulphonyl having in each case 1 to 5 halogen atoms; represent —SO₂NR¹²R¹³, —C(═X)R¹⁴, —Si(R¹⁵)₃, C₂-C₄-alkenylene-Si(R¹⁵)₃, C₂-C₄-alkynylene-Si(R¹⁵)₃, —NR¹²_{R}¹³, —CH₂NR¹²R¹³.
R⁹, R¹⁰ and R¹¹ independently of one another particularly preferably represent hydrogen, fluorine, chlorine, bromine, iodine, cyano, nitro, methyl, ethyl, n- or isopropyl, n-, iso-, sec- or tert-butyl, allyl, propargyl, methoxy, ethoxy, n- or isopropoxy, n-, iso-, sec- or tert-butoxy, methylthio, ethylthio, n- or isopropylthio, n-, iso-, sec- or tert-butylthio, methylsulphinyl, ethylsulphinyl, n- or isopropylsulphinyl, n-, iso-, sec- or tert-butylsulphinyl, methylsulphonyl, ethylsulphonyl, n- or isopropylsulphonyl, n-, iso-, sec- or tert-butylsulphonyl, cyclopropyl, cyclopentyl, cyclohexyl, trifluoromethyl, difluoromethyl, trichloromethyl, trifluoroethyl, trifluoromethoxy, difluoromethoxy, trichloromethoxy, difluoromethylthio, difluorochloromethylthio, trifluoromethylthio, trifluoromethylsulphinyl, trifluoromethylsulphonyl, —SO₂NMe₂, —C(═X)R¹⁴, —Si(R¹⁵)₃, —CH═CH—Si(R¹⁵)₃, —CH₂—CH═CH—Si(R¹⁵)₃, —CH═CH—CH₂—Si(R¹⁵)₃, —C≡C—Si(R¹⁵)₃, —CH₂—C≡C—Si(R¹⁵)₃, —C≡C—CH₂—Si(R¹⁵)₃, —CH₂C≡C—CH₂—Si(R¹⁵)₃, —NR¹²R¹³, —CH₂—NR¹²R¹³.
R⁹, R¹° and R^{H} independently of one another very particularly preferably represent hydrogen, fluorine, chlorine, bromine, iodine, cyano, methyl, ethyl, n- or isopropyl, allyl, propargyl, methoxy, ethoxy, n- or isopropoxy, methylthio, ethylthio, n- or isopropylthio, cyclopropyl, cyclopentyl, cyclohexyl, trifluoromethyl, difluoromethyl, trifluoroethyl, trifluoromethoxy, difluoromethoxy, difluoromethylthio, difluorochloromethylthio, trifluoromethylthio, trifluoromethylsulphinyl, trifluoromethylsulphonyl, —SO₂NMe₂, —C(═X)R¹⁴, —Si(R¹⁵)₃, —CH═CH—Si(R¹⁵)₃, —CH₂——CH═CH—Si(R¹⁵)₃, —CH═CH—CH₂—Si(R¹⁵)₃, C≡C—Si(R¹⁵)₃, —CH₂—C≡C—Si(R¹⁵)₃, —C≡C—CH₂—Si(R¹⁵)₃, ——CH₂—C≡C—CH₂—Si(R¹⁵)₃, —NR¹²R¹³, —CH₂—NR¹²R¹³.
R⁹, R¹⁰ and R¹¹ independently of one another especially preferably represent hydrogen, fluorine, chlorine, bromine, iodine, methyl, allyl, propargyl, methoxy, methylthio, cyclopropyl, cyclopentyl, cyclohexyl, trifluoromethyl, difluoromethyl, trifluoroethyl, trifluoromethoxy, difluoromethoxy, difluoromethylthio, trifluoromethylthio, —SO₂NMe₂, —C(═X)R¹⁴, —NR¹²R¹³, —CH₂—NR¹²R¹³.
R¹² preferably represents hydrogen, methyl, ethyl, n- or isopropyl, n-, iso-, sec- or tert-butyl or —C(═X)R¹⁴.
R¹² particularly preferably represents hydrogen, methyl, ethyl, n- or isopropyl or —C(═X)R¹⁴.
R¹² very particularly preferably represents hydrogen or methyl.
R¹³ preferably represents hydrogen, methyl, ethyl, n- or isopropyl, n-, iso-, sec- or tert-butyl or —C(═X)R¹⁴.
R¹³ particularly preferably represents hydrogen, methyl, ethyl, n- or isopropyl or —C(═X)R¹⁴.
R¹³ very particularly preferably represents hydrogen or methyl.

R¹² and R¹³ furthermore together with the nitrogen atom to which they are attached preferably form a saturated heterocycle which is optionally mono- or polysubstituted by identical or different substituents from the group consisting of halogen and C₁-C₄-alkyl and has 5 or 6 ring atoms, where the heterocycle may contain 1 or 2 further non-adjacent heteroatoms from the group consisting of oxygen, sulphur and NR⁷.
R¹² and R¹³ furthermore together with the nitrogen atom to which they are attached particularly preferably form a saturated heterocycle from the group consisting of morpholine, thiomorpholine and piperazine which is optionally mono- to tetrasubstituted by identical or different substituents from the group consisting of fluorine, chlorine, bromine and methyl, where the piperazine may be substituted on the second nitrogen atom by R⁷.
X preferably represents O (oxygen).
X also preferably represents S (sulphur).
R¹⁴ preferably represents hydrogen, methyl, ethyl, n- or isopropyl, n-, iso-, sec- or tert-butyl, methoxy, ethoxy, n- or isopropoxy, n-, iso-, sec- or tert-butoxy or —NR¹⁶R¹⁷.
R¹⁴ particularly preferably represents hydrogen, methyl, ethyl, n- or isopropyl, methoxy, ethoxy, n- or isopropoxy or —NR¹⁶R¹⁷.
R¹⁴ very particularly preferably represents hydrogen, methyl, ethyl, methoxy, ethoxy or —NR¹⁶R¹⁷.
R¹⁵ preferably represents C₁-C₆-alkyl, C₁-C₆-alkoxy, C₁-C₃-alkoxy-C₁-C₃-alkyl, where the three radicals R¹⁵ may each be identical or different.
R¹⁵ particularly preferably represents methyl, ethyl, methoxy, ethoxy, methoxymethyl, ethoxymethyl, methoxyethyl, ethoxyethyl, methylthiomethyl, ethylthiomethyl, methylthioethyl or ethylthioethyl, where the three radicals R¹⁵ may each be identical or different.
R¹⁵ very particularly preferably represents methyl, methoxy, methoxymethyl or methylthiomethyl, where the three radicals R¹⁵ may each be identical or different.
R¹⁵ especially preferably represents methyl.
R¹⁶ preferably represents hydrogen, methyl, ethyl, n- or isopropyl, n-, iso-, sec- or tert-butyl.
R¹⁶ particularly preferably represents hydrogen, methyl, ethyl, n- or isopropyl.
R¹⁶ very particularly preferably represents hydrogen or methyl.
R¹⁷ preferably represents hydrogen, methyl, ethyl, n- or isopropyl, n-, iso-, sec- or tert-butyl.
R¹⁷ particularly preferably represents hydrogen, methyl, ethyl, n- or isopropyl.
R¹⁷ very particularly preferably represents hydrogen or methyl.
R¹⁶ and R¹⁷ furthermore together with the nitrogen atom to which they are attached preferably form a saturated heterocycle which is optionally mono- or polysubstituted by identical or different substituents from the group consisting of halogen and C₁-C₄-alkyl and has 5 or 6 ring atoms, where the heterocycle may contain 1 or 2 further non-adjacent heteroatoms from the group consisting of oxygen, sulphur and NR⁷.
R¹⁶ and R¹⁷ furthermore together with the nitrogen atom to which they are attached particularly preferably form a saturated heterocycle from the group consisting of morpholine, thiomorpholine and piperazine which is optionally mono- to tetrasubstituted by identical or different substituents from the group consisting of fluorine, chlorine, bromine and methyl, where the piperazine may be substituted on the second nitrogen atom by R⁷.
A preferably represents one of the radicals A1, A2, A3, A4, A5, A6, A9, A10, A11, A12 or A17.
A particularly preferably represents one of the radicals A1, A2, A4, A5, A6, A9, A11, A16, A17.
A very particularly preferably represents the radical A1.
A furthermore very particularly preferably represents the radical A2.
A furthermore very particularly preferably represents the radical A4.
A furthermore very particularly preferably represents the radical A5.
A furthermore very particularly preferably represents the radical A6.
A furthermore very particularly preferably represents the radical A9.
A furthermore very particularly preferably represents the radical A11.
A furthermore very particularly preferably represents the radical A16.
A furthermore very particularly preferably represents the radical A17.
R¹⁸ preferably represents hydrogen, cyano, fluorine, chlorine, bromine, iodine, methyl, ethyl, isopropyl, methoxy, ethoxy, methylthio, ethylthio, cyclopropyl, C₁-C₂-haloalkyl, C₁-C₂-haloalkoxy having in each case 1 to 5 fluorine, chlorine and/or bromine atoms, trifluoromethylthio, difluoromethylthio, aminocarbonyl, aminocarbonylmethyl or aminocarbonylethyl.
R¹⁸ particularly preferably represents hydrogen, fluorine, chlorine, bromine, iodine, methyl, ethyl, isopropyl, monofluoromethyl, monofluoroethyl, difluoromethyl, trifluoromethyl, difluorochloromethyl, trichloromethyl, dichloromethyl, cyclopropyl, methoxy, ethoxy, trifluoromethoxy, trichloromethoxy, methylthio, ethylthio, trifluoromethylthio or difluoromethylthio.
R¹⁸ very particularly preferably represents hydrogen, fluorine, chlorine, bromine, iodine, methyl, isopropyl, monofluoromethyl, monofluoroethyl, difluoromethyl, trifluoromethyl, difluoro-chloromethyl or trichloromethyl.
R¹⁸ especially preferably represents methyl, difluoromethyl, trifluoromethyl or 1-fluoroethyl.
R¹⁹ preferably represents hydrogen, fluorine, chlorine, bromine, iodine, methyl, ethyl, methoxy, ethoxy, methylthio or ethylthio.
R¹⁹ particularly preferably represents hydrogen, fluorine, chlorine, bromine, iodine or methyl.
R¹⁹ very particularly preferably represents hydrogen, fluorine, chlorine or methyl.
R²⁰ preferably represents hydrogen, methyl, ethyl, n-propyl, isopropyl, C₁-C₂-haloalkyl having 1 to 5 fluorine, chlorine and/or bromine atoms, hydroxymethyl, hydroxyethyl, cyclopropyl, cyclopentyl, cyclohexyl or phenyl.
R²⁰ particularly preferably represents hydrogen, methyl, ethyl, isopropyl, trifluoromethyl, difluoromethyl, hydroxymethyl, hydroxyethyl or phenyl.
R²⁰ very particularly preferably represents hydrogen, methyl, trifluoromethyl or phenyl.
R²⁰ especially preferably represents methyl.
R²¹ and R²² independently of one another preferably represent hydrogen, fluorine, chlorine, bromine, methyl, ethyl or C₁-C₂-haloalkyl having 1 to 5 fluorine, chlorine and/or bromine atoms.
R²¹ and R²² independently of one another particularly preferably represent hydrogen, fluorine, chlorine, bromine, methyl, ethyl, difluoromethyl, trifluoromethyl, difluorochloromethyl or trichloromethyl.
R²¹ and R²² independently of one another very particularly preferably represent hydrogen, fluorine, chlorine, bromine, methyl, ethyl, difluoromethyl, trifluoromethyl or trichloromethyl.
R²¹ and R²² especially preferably each represent hydrogen.
R²³ preferably represents fluorine, chlorine, bromine, cyano, methyl, ethyl, C₁-C₂-haloalkyl or C₁-C₂-haloalkoxy having in each case 1 to 5 fluorine, chlorine and/or bromine atoms.
R²³ particularly preferably represents fluorine, chlorine, bromine, cyano, methyl, trifluoromethyl, trifluoromethoxy, difluoromethoxy, difluorochloromethoxy or trichloromethoxy.
R²³ very particularly preferably represents fluorine, chlorine, bromine, iodine, methyl, trifluoromethyl or trifluoromethoxy.
R²³ especially preferably represents methyl or trifluoromethyl.
R²⁴ and R²⁵ independently of one another preferably represent hydrogen, fluorine, chlorine, bromine, methyl, ethyl or C₁-C₂-haloalkyl having 1 to 5 fluorine, chlorine and/or bromine atoms.
R²⁴ and R²⁵ independently of one another particularly preferably represent hydrogen, fluorine, chlorine, bromine, methyl, ethyl, difluoromethyl, trifluoromethyl, difluorochloromethyl or trichloromethyl.
R²⁴ and R²⁵ independently of one another very particularly preferably represent hydrogen, fluorine, chlorine, bromine, methyl, ethyl, difluoromethyl, trifluoromethyl or trichloromethyl.
R²⁴ and R²⁵ especially preferably each represent hydrogen.
R26 preferably represents hydrogen, fluorine, chlorine, bromine, iodine, methyl, ethyl or C₁-C₂-haloalkyl having 1 to 5 fluorine, chlorine and/or bromine atoms.
R²⁶ particularly preferably represents hydrogen, fluorine, chlorine, bromine, iodine, methyl or trifluoromethyl.
R²⁶ very particularly preferably represents fluorine, chlorine, bromine, iodine, methyl or trifluoromethyl.
R²⁷ preferably represents hydrogen, fluorine, chlorine, bromine, iodine, hydroxyl, cyano, C₁-C₂-haloalkyl, C₁-C₂-haloalkoxy or C₁-C₂-haloalkylthio having in each case 1 to 5 fluorine, chlorine and/or bromine atoms.
R²⁷ particularly preferably represents hydrogen, fluorine, chlorine, bromine, iodine, hydroxyl, cyano, methyl, ethyl, n-propyl, isopropyl, n-butyl, isobutyl, sec-butyl, tert-butyl, difluoromethyl, trifluoromethyl, difluorochloromethyl, trichloromethyl, trifluoromethoxy, difluoromethoxy, difluorochloromethoxy, trichloromethoxy, trifluoromethylthio, difluoromethylthio, difluorochloromethylthio or trichloromethylthio.
R²⁷ very particularly preferably represents hydrogen, fluorine, chlorine, bromine, iodine, methyl, difluoromethyl, trifluoromethyl or trichloromethyl.
R²⁷ especially preferably represents iodine, methyl, difluoromethyl or trifluoromethyl.
R²⁸ preferably represents fluorine, chlorine, bromine, iodine, hydroxyl, cyano, methoxy, ethoxy, methylthio, ethylthio, difluoromethylthio, trifluoromethylthio, C₁-C₂-haloalkyl or C₁-C₂-haloalkoxy having in each case 1 to 5 fluorine, chlorine and/or bromine atoms.
R²⁸ particularly preferably represents fluorine, chlorine, bromine, iodine, hydroxyl, cyano, methyl, ethyl, n-propyl, isopropyl, n-butyl, isobutyl, sec-butyl, tert-butyl, trifluoromethyl, difluoromethyl, difluorochloromethyl, trichloromethyl, methoxy, ethoxy, methylthio, ethylthio, difluoromethylthio, trifluoromethylthio, trifluoromethoxy, difluoromethoxy, difluorochloromethoxy or trichloromethoxy.
R²⁸ particularly preferably represents fluorine, chlorine, bromine, iodine, methyl, trifluoromethyl, difluoromethyl or trichloromethyl.
R²⁹ preferably represents hydrogen, fluorine, chlorine, bromine, iodine, cyano, methoxy, ethoxy, methylthio, ethylthio, C₁-C₂-haloalkyl or C₁-C₂-haloalkoxy having in each case 1 to 5 fluorine, chlorine and/or bromine atoms, C₁-C₂-alkylsulphinyl or C₁-C₂-alkylsulphonyl.
R²⁹ particularly preferably represents hydrogen, fluorine, chlorine, bromine, iodine, cyano, n-propyl, isopropyl, n-butyl, isobutyl, sec-butyl, tert-butyl, trifluoromethyl, difluoromethyl, difluorochloromethyl, trichloromethyl, methoxy, ethoxy, methylthio, ethylthio, trifluoromethoxy, difluoromethoxy, difluorochloromethoxy, trichloromethoxy, methylsulphinyl or methylsulphonyl.
R²⁹ very particularly preferably represents hydrogen, fluorine, chlorine, bromine, iodine, n-propyl, isopropyl, n-butyl, isobutyl, sec-butyl, tert-butyl, trifluoromethyl, difluoromethyl, trichloromethyl, methylsulphinyl or methylsulphonyl.
R²⁹ especially preferably represents hydrogen.
R³⁰ preferably represents methyl, ethyl or C₁-C₂-haloalkyl having 1 to 5 fluorine, chlorine and/or bromine atoms.
R³⁰ particularly preferably represents methyl, ethyl, trifluoromethyl, difluoromethyl, difluorochloromethyl or trichloromethyl.
R³¹ preferably represents methyl or ethyl.
R³¹ particularly preferably represents methyl.
Q¹ preferably represents S (sulphur), SO₂ or CH₂.
Q¹ particularly preferably represents S (sulphur) or CH₂.
Q¹ very particularly preferably represents S (sulphur).
p preferably represents 0 or 1.
p particularly preferably represents 0.
R³² preferably represents methyl, ethyl or C₁-C₂-haloalkyl having 1 to 5 fluorine, chlorine and/or bromine atoms.
R³² particularly preferably represents methyl, ethyl, trifluoromethyl, difluoromethyl, difluorochloromethyl or trichloromethyl.
R³² very particularly preferably represents methyl, trifluoromethyl, difluoromethyl or trichloromethyl.

R³³ preferably represents methyl, ethyl or C₁-C₂-haloalkyl having 1 to 5 fluorine, chlorine and/or bromine atoms.

R³³ particularly preferably represents methyl, ethyl, trifluoromethyl, difluoromethyl, difluorochloromethyl or trichloromethyl.
R³³ very particularly preferably represents methyl, trifluoromethyl, difluoromethyl or trichloromethyl.
R³⁴ and R³⁵ independently of one another preferably represent hydrogen, fluorine, chlorine, bromine, amino, methyl, ethyl or C₁-C₂-haloalkyl having 1 to 5 fluorine, chlorine and/or bromine atoms.
R³⁴ and R³⁵ independently of one another particularly preferably represent hydrogen, fluorine, chlorine, bromine, methyl, ethyl, trifluoromethyl, difluoromethyl, difluorochloromethyl or trichloromethyl.
R³⁴ and R³⁵ independently of one another very particularly preferably represent hydrogen, fluorine, chlorine, bromine, methyl, trifluoromethyl, difluoromethyl or trichloromethyl.
R³⁴ and R³⁵ especially preferably each represent hydrogen.
R³⁶ preferably represents hydrogen, fluorine, chlorine, bromine, iodine, methyl, ethyl or C₁-C₂-haloalkyl having 1 to 5 fluorine, chlorine and/or bromine atoms.
R³⁶ particularly preferably represents hydrogen, fluorine, chlorine, bromine, iodine, methyl, ethyl, trifluoromethyl, difluoromethyl, difluorochloromethyl or trichloromethyl.
R³⁶ very particularly preferably represents hydrogen, fluorine, chlorine, bromine, iodine, methyl, trifluoromethyl, difluoromethyl or trichloromethyl.
R³⁶ especially preferably represents methyl.
R³⁷ and R³⁸ independently of one another preferably represent hydrogen, fluorine, chlorine, bromine, amino, nitro, methyl, ethyl or C₁-C₂-haloalkyl having 1 to 5 fluorine, chlorine and/or bromine atoms.
R³⁷ and R³⁸ independently of one another particularly preferably represent hydrogen, fluorine, chlorine, bromine, nitro, methyl, ethyl, trifluoromethyl, difluoromethyl, difluorochloromethyl or trichloromethyl.
R³⁷ and R³⁸ independently of one another very particularly preferably represent hydrogen, fluorine, chlorine, bromine, methyl, trifluoromethyl, difluoromethyl or trichloromethyl.
R³⁷ and R³⁸ especially preferably each represent hydrogen.
R³⁹ preferably represents hydrogen, fluorine, chlorine, bromine, methyl, ethyl or C₁-C₂-haloalkyl having 1 to 5 fluorine, chlorine and/or bromine atoms,
R³⁹ particularly preferably represents hydrogen, fluorine, chlorine, bromine, methyl, ethyl, trifluoromethyl, difluoromethyl, difluorochloromethyl or trichloromethyl.
R³⁹ very particularly preferably represents hydrogen, fluorine, chlorine, bromine, methyl, trifluoromethyl, difluoromethyl or trichloromethyl.
R³⁹ especially preferably represents methyl.
R⁴⁰ preferably represents hydrogen, fluorine, chlorine, bromine, amino, C₁-C₄-alkylamino, di(C₁-C₄-alkyl)amino, cyano, methyl, ethyl or C₁-C₂-haloalkyl having 1 to 5 fluorine, chlorine and/or bromine atoms.
R⁴⁰ particularly preferably represents hydrogen, fluorine, chlorine, bromine, amino, methylamino, dimethylamino, cyano, methyl, ethyl, trifluoromethyl, difluoromethyl, difluorochloromethyl or trichloromethyl.
R⁴⁰ very particularly preferably represents hydrogen, fluorine, chlorine, bromine, amino, methylamino, dimethylamino, methyl, trifluoromethyl, difluoromethyl or trichloromethyl.
R⁴⁰ especially preferably represents amino, methylamino, dimethylamino, methyl or trifluoromethyl.
R⁴¹ preferably represents fluorine, chlorine, bromine, methyl, ethyl or C₁-C₂-haloalkyl having 1 to 5 fluorine, chlorine and/or bromine atoms.
R⁴¹ particularly preferably represents fluorine, chlorine, bromine, methyl, ethyl, trifluoromethyl, difluoromethyl, difluorochloromethyl or trichloromethyl.
R⁴¹ very particularly preferably represents fluorine, chlorine, bromine, methyl, trifluoromethyl, difluoromethyl or trichloromethyl.
R⁴¹ especially preferably represents methyl, trifluoromethyl or difluoromethyl.
R⁴² preferably represents hydrogen, fluorine, chlorine, bromine, amino, C₁-C₄-alkylamino, di(C₁-C₄-alkyl)amino, cyano, methyl, ethyl or C₁-C₂-haloalkyl having 1 to 5 fluorine, chlorine and/or bromine atoms.
R⁴² particularly preferably represents hydrogen, fluorine, chlorine, bromine, amino, methylamino, dimethylamino, cyano, methyl, ethyl, trifluoromethyl, difluoromethyl, difluorochloromethyl or trichloromethyl.
R⁴² very particularly preferably represents hydrogen, fluorine, chlorine, bromine, amino, methylamino, dimethylamino, methyl, trifluoromethyl, difluoromethyl or trichloromethyl.
R⁴² especially preferably represents amino, methylamino, dimethylamino, methyl or trifluoromethyl.
R⁴³ preferably represents fluorine, chlorine, bromine, methyl, ethyl or C₁-C₂-haloalkyl having 1 to 5 fluorine, chlorine and/or bromine atoms.
R⁴³ particularly preferably represents fluorine, chlorine, bromine, methyl, ethyl, trifluoromethyl, difluoromethyl, difluorochloromethyl or trichloromethyl.
R⁴³ very particularly preferably represents fluorine, chlorine, bromine, methyl, trifluoromethyl, difluoromethyl or trichloromethyl.
R⁴³ especially preferably represents methyl, trifluoromethyl or difluoromethyl.
R⁴⁴ preferably represents fluorine, chlorine, bromine, methyl, ethyl or C₁-C₂-haloalkyl having 1 to 5 fluorine, chlorine and/or bromine atoms.
R⁴⁴ particularly preferably represents fluorine, chlorine, bromine, methyl, ethyl, trifluoromethyl, difluoromethyl, difluorochloromethyl or trichloromethyl.
R⁴⁴ very particularly preferably represents fluorine, chlorine, bromine, methyl, trifluoromethyl, difluoromethyl or trichloromethyl.
R⁴⁵ preferably represents hydrogen, methyl or ethyl.
R⁴⁵ particularly preferably represents methyl.
R⁴⁶ preferably represents fluorine, chlorine, bromine, methyl or ethyl.
R⁴⁶ particularly preferably represents fluorine, chlorine or methyl.
R⁴⁷ preferably represents methyl, ethyl or C₁-C₂-haloalkyl having 1 to 5 fluorine, chlorine and/or bromine atoms.
R⁴⁷ particularly preferably represents methyl, ethyl, trifluoromethyl, difluoromethyl, difluorochloromethyl or trichloromethyl.
R⁴⁷ very particularly preferably represents methyl, trifluoromethyl, difluoromethyl or trichloromethyl.
R⁴⁷ especially preferably represents methyl or trifluoromethyl.
R⁴⁸ preferably represents hydrogen, fluorine, chlorine, bromine, methyl, ethyl or C₁-C₂-haloalkyl having 1 to 5 fluorine, chlorine and/or bromine atoms.
R⁴⁸ particularly preferably represents hydrogen, fluorine, chlorine, bromine, methyl or trifluoromethyl.
R⁴⁹ preferably represents fluorine, chlorine, bromine, iodine, hydroxyl, C₁-C₄-alkyl, methoxy, ethoxy, methylthio, ethylthio, difluoromethylthio, trifluoromethylthio, C₁-C₂-haloalkyl or C₁-C₂-haloalkoxy having in each case 1 to 5 fluorine, chlorine and/or bromine atoms.
R⁴⁹ particularly preferably represents fluorine, chlorine, bromine, iodine, methyl, ethyl, n-propyl, isopropyl, n-butyl, isobutyl, sec-butyl, tert-butyl, trifluoromethyl, difluoromethyl, difluorochloromethyl or trichloromethyl.
R⁴⁹ very particularly preferably represents fluorine, chlorine, bromine, iodine, methyl, trifluoromethyl, difluoromethyl or trichloromethyl.
R⁵⁰ preferably represents methyl, ethyl, n-propyl or isopropyl.
R⁵⁰ particularly preferably represents methyl or ethyl.

Preference is given to those compounds of the formula (I) in which all radicals each have the preferred meanings mentioned above.

Particular preference is given to those compounds of the formula (I) in which all radicals each have the particularly preferred meanings mentioned above.

Preference is furthermore given to compounds of the formula (I-b)

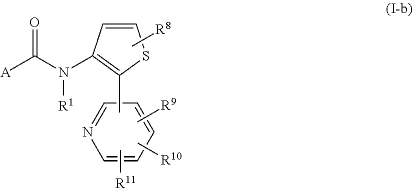

in which R¹, R⁸, R⁹, R¹⁰, R¹¹ and A are as defined above.

Preference is furthermore given to compounds of the formula (I-c)

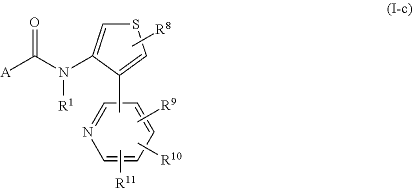

in which R¹, R⁸, R⁹, R¹⁰, R¹¹ and A are as defined above.

Preference is furthermore given to compounds of the formula (I-d)

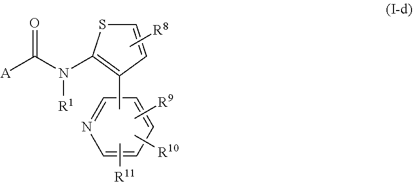

in which R¹, R⁸, R⁹, R¹⁰, R¹¹ and A are as defined above.

Preference is furthermore given to compounds of the formula (I-e)

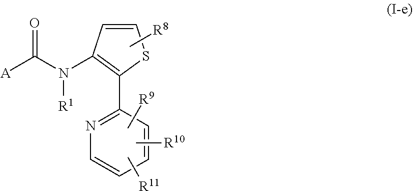

in which R¹, R⁸, R⁹, R¹⁰, R¹¹ and A n are as defined above.

Preference is furthermore given to compounds of the formula (I-f)

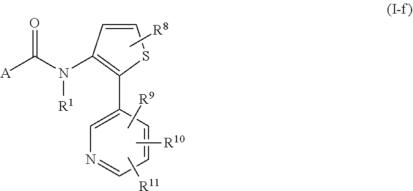

in which R¹, R⁸, R⁹, R¹⁰, R¹¹ and A are as defined above.

Preference is furthermore given to compounds of the formula (I-g)

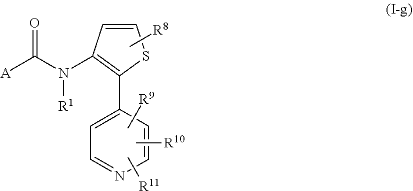

in which R¹, R⁸, R⁹, R¹⁰, R¹¹ and A are as defined above.

Preference is furthermore given to compounds of the formula (I-h)

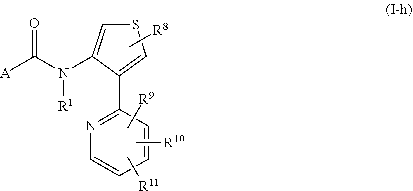

in which R¹, R⁸, R⁹, R¹⁰, R¹¹ and A are as defined above.

Preference is furthermore given to compounds of the formula (I-i)

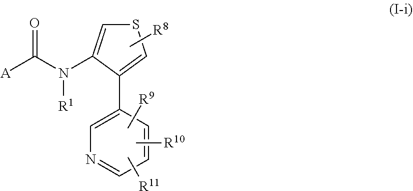

in which R¹, R⁸, R⁹, R¹⁰, R¹¹ and A are as defined above.

Preference is furthermore given to compounds of the formula (I-j)

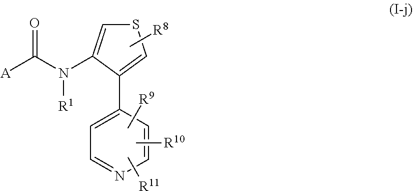

in which R¹, R⁸, R⁹, R¹⁰, R¹¹ and A are as defined above.

Preference is furthermore given to compounds of the formula (I-k)

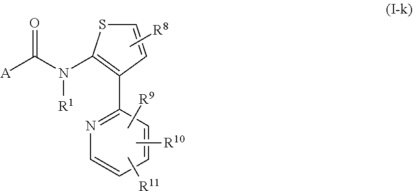

in which R¹, R⁸, R⁹, R¹⁰, R¹¹ and A are as defined above.

Preference is furthermore given to compounds of the formula (I-f)

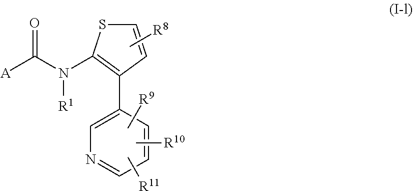

in which R¹, R⁸, R⁹, R¹⁰, R¹¹ and A are as defined above.

Preference is furthermore given to compounds of the formula (I-m)

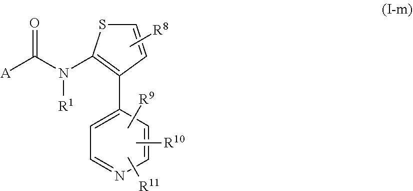

in which R¹, R⁸, R⁹, R¹⁰, R¹¹ and A are as defined above.

Preference is likewise given to compounds of the formulae (I) and (I-b) to (I-m), in which R¹ represents hydrogen.

Preference is likewise given to compounds of the formulae (I) and (I-b) to (I-m), in which R⁸ represents hydrogen.

Preference is likewise given to compounds of the formulae (I) and (I-b) to (I-m), in which R⁹ represents hydrogen.

Preference is likewise given to compounds of the formulae (I) and (I-b) to (I-m), in which R⁹ and R¹⁰ both represent hydrogen.

Using, for example, 2-chloronicotinoyl chloride and 2-(5-bromopyridin-2-yl)thiophene-3-amine as starting materials and a base, the course of the process (a) according to the invention can be illustrated by the reaction equation below:

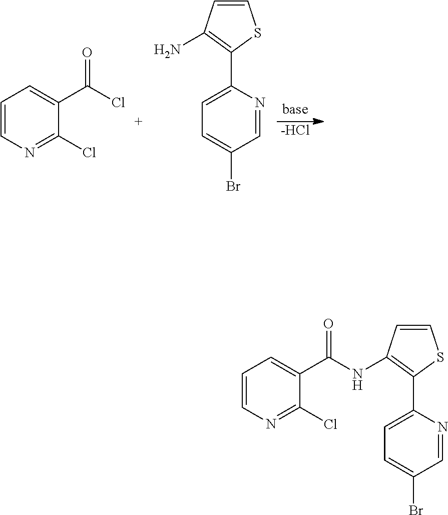

The formula (II) provides a general definition of the carbonyl halides required as starting materials for carrying out the process (a) according to the invention. In this formula (II), A preferably, particularly preferably, very particularly preferably and especially preferably has those meanings which have already been mentioned in connection with the description of the compounds of the formula (I) according to the invention as being preferred, particularly preferred, etc., for this radical. X¹ preferably represents fluorine, chlorine or hydroxyl, particularly preferably chlorine or hydroxyl.

The carbonyl halides of the formula (II) are known and/or can be prepared by known processes (cf., for example, EP-A 0 545 099, JP-A 01-290662 and U.S. Pat. No. 5,093,347).

The formula (III) provides a general definition of the amines furthermore required as starting materials for carrying out the process (a) according to the invention. In this formula (III), R¹, R⁸, R⁹, R¹⁰ and R¹¹ preferably, particularly preferably, very particularly preferably and especially preferably have those meanings which have already been mentioned in connection with the description of the compounds of the formula (I) according to the invention as being preferred, particularly preferred, etc., for this radical.

The amines of the formula (III) are novel. They can be prepared by (c) reacting dioxazaborocane derivatives of the formula (V)

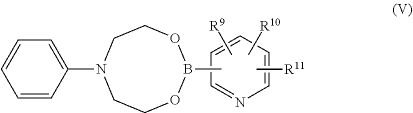

in which R⁹, R¹⁰ and R¹¹ are as defined above,
with carbamates of the formula (VI)

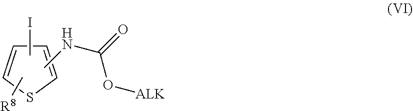

in which
R⁸ is as defined above,
ALK represents alkyl, preferably C₁-C₆-alkyl, particularly preferably C₁-C₄-alkyl, very particularly preferably methyl, isopropyl or tert-butyl, in particular tert-butyl,
in the presence of a catalyst [for example bis(triphenylphosphine)palladium(II) chloride], in the presence of a base (for example sodium carbonate) and, if appropriate, in the presence of a diluent (for example a mixture of water and dimethyl sulphoxide),
and reacting the resulting amines of the formula (III-a)

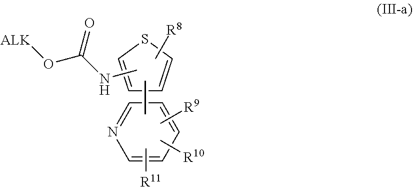

in which R⁸, R⁹, R¹⁰, R¹¹ and ALK are as defined above
in the presence of an acid (for example trifluoroacetic acid) and, if appropriate, in the presence of a diluent (for example dichloromethane),
and, if appropriate, reacting the resulting amines of the formula (III-b)

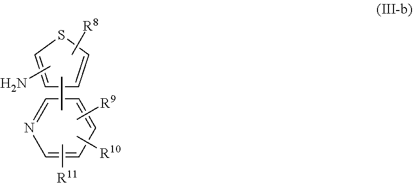

in which R⁸, R⁹, R¹⁰ and R¹¹ are as defined above with halides of the formula (IV)

R^{1-B}-Hal (IV)
in which R^{1-B} and Hal are as defined above
in the presence of a base and in the presence of a diluent [for this last reaction step, the conditions of process (b) according to the invention apply in a corresponding manner].

Using N-[2-(5-bromopyridin-2-yl)-3-thienyl]-2-chloronicotinamide and acetyl chloride as starting materials, the course of the process (b) according to the invention can be illustrated by the formula scheme below:

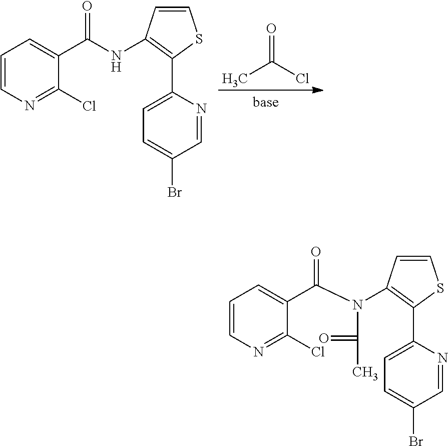

The formula (I-a) provides a general definition of the thienylpyridylcarboxamides required as starting materials for carrying out the process (b) according to the invention. In this formula (I-a), R⁸, R⁹, R¹⁰, R¹¹ and A preferably, particularly preferably, very particularly preferably and especially preferably have those meanings which have already been mentioned in connection with the description of the compounds of the formula (I) according to the invention as being preferred, particularly preferred, etc., for these radicals.

The compounds of the formula (I-a) are compounds according to the invention and can be prepared according to process (a).

The formula (IV) provides a general definition of the halides furthermore required as starting materials for carrying out the process (b) according to the invention. In this formula (IV), R^{1-B} preferably, particularly preferably, very particularly preferably and especially preferably has those meanings which have already been mentioned above as being preferred, particularly preferred, etc., for the radical R¹, where R^{1-B} is never hydrogen. Hal represents chlorine, bromine or iodine.

Halides of the formula (IV) are known.

Using tert-butyl(2-iodo-3-thienyl)carbamate and 2-(5-bromopyridin-2-yl)-6-phenyl-1,3,6,2-dioxaza-borocane as starting materials and an acid, the course of the process (c) according to the invention can be illustrated by the formula scheme below:

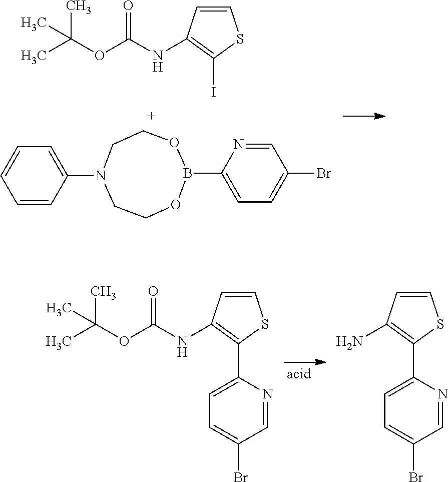

The formula (V) provides a general definition of the dioxazaborocane derivatives required as starting materials for carrying out the process (c) according to the invention. In this formula (V), R⁹, R¹⁰ and R¹¹ preferably, particularly preferably, very particularly preferably and especially preferably have those meanings which have already been mentioned in connection with the description of the compounds of the formula (I) according to the invention as being preferred, particularly preferred, etc., for these radicals.

The dioxazaborocane derivatives of the formula (V) are novel. They can be obtained, for example, by (d) reacting iodinated pyridine derivatives of the formula (VII)

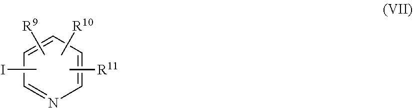

in which R⁹, R¹⁰ and R¹¹ are as defined above
in the presence of a diluent (for example tetrahydrofuran) initially with trialkyl borates (for example triisopropyl borate) and then with N-phenyldiethanolamine (see also the Preparation Examples).

The formula (VI) provides a general definition of the carbamates furthermore required as starting materials for carrying out the process (c) according to the invention. In this formula (VI), R⁸ preferably, particularly preferably, very particularly preferably and especially preferably has those meanings which have already been mentioned above as being preferred, particularly preferred, etc., for the radical R¹.

Carbamates of the formula (VI) are novel. They are obtained, for example, when (e) carbamates of the formula (VIII)

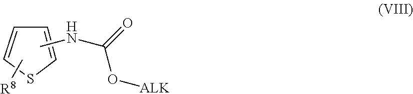

in which R⁸ and ALK are as defined above
are iodinated in the presence of a base (for example n-butyllithium) and in the presence of a diluent (for example tetrahydrofuran) (see also the Preparation Examples).

The formula (VII) provides a general definition of the iodinated pyridine derivatives required as starting materials for carrying out the process (d) according to the invention. In this formula (VII), R⁹, R¹⁰ and R¹¹ preferably, particularly preferably, very particularly preferably and especially preferably have those meanings which have already been mentioned in connection with the description of the compounds of the formula (I) as being preferred, particularly preferred, etc., for these radicals.

Iodinated pyridine derivatives of the formula (VII) are known or can be obtained in a known manner.

Triisopropyl borate and N-phenyldiethanolamine, which are furthermore required as starting materials for carrying out the process (d) according to the invention, are known chemicals for synthesis.

The formula (VIII) provides a general definition of the carbamates required as starting materials for carrying out the process (e) according to the invention. In this formula (VIII), R⁸ preferably, particularly preferably, very particularly preferably and especially preferably has those meanings which have already been mentioned above as being preferred, particularly preferred, etc., for the radical R¹.

Carbamates of the formula (VIII) are known or can be obtained in a known manner.

Suitable diluents for carrying out the process (a) according to the invention are all inert organic solvents.

These preferably include aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as diethyl ether, diisopropyl ether, methyl t-butyl ether, methyl-t-amyl ether, dioxane, tetrahydrofuran, 1,2-dimethoxyethane, 1,2-diethoxyethane or anisole; ketones, such as acetone, butanone, methyl isobutyl ketone or cyclohexanone; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone or hexamethylphosphoric triamide; mixtures thereof with water or pure water.

The process (a) according to the invention is, if appropriate, carried out in the presence of a suitable acid acceptor. Suitable acid acceptors are all customary inorganic or organic bases. These preferably include alkaline earth metal or alkali metal hydrides, hydroxides, amides, alkoxides, acetates, carbonates or bicarbonates, such as, for example, sodium hydride, sodium amide, lithium diisopropylamide, sodium methoxide, sodium ethoxide, potassium tert-butoxide, sodium hydroxide, potassium hydroxide, sodium acetate, sodium carbonate, potassium carbonate, potassium bicarbonate, sodium bicarbonate or ammonium carbonate, and also tertiary amines, such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU).

The process (a) according to the invention is, if appropriate, carried out in the presence of a suitable coupling agent (if X⁵ represents hydroxyl). Suitable coupling agents are all customary carbonyl activators. These preferably include N-[3-(dimethylamino)propyl]-N′-ethylcarbodiimide hydrochloride, N,N′-di-sec-butylcarbodiimide, N,N′-dicyclohexylcarbodiimide, N,N′-diisopropylcarbodiimide, 1-(3-(dimethylamino)propyl)-3-ethylcarbodiimide methiodide, 2-bromo-3-ethyl-4-methylthiazolium tetrafluoroborate, N,N-bis[2-oxo-3-oxazolidinyl]phosphorodiamidic chloride, chlorotripyrrolidinophosphonium hexafluorophosphate, bromotripyrrolidinophosphonium hexafluorophosphate, O-(1H-benzotriazol-1-yloxy)tris(dimethylamino)phosphonium hexafluorophosphate, O-(1H-benzotriazol-1-yl)-N,N,N′,N′-tetramethyluronium hexafluorophosphate, O-(1H-benzotriazol-1-yl)-N,N,N′,N′-bis(tetramethylene)uronium hexafluorophosphate, O-(1H-benzotriazol-1-yl)-N,N,N,N′-bis(tetramethylene)uronium tetrafluoroborate, N,N,N′,N′-bis(tetramethylene)chlorouronium tetrafluoroborate, O-(7-azabenzotriazol-1-yl)-N,N,N,N-tetramethyluronium hexafluorophosphate and 1-hydroxybenzotriazole. These reagents can be employed separately, but also in combination.

When carrying out the process (a) according to the invention, the reaction temperatures can be varied within a relatively wide range. In general, the process is carried out at temperatures of from 0° C. to 150° C., preferably at temperatures of from 20° C. to 110° C.

For carrying out the process (a) according to the invention for preparing the compounds of the formula (I), in general from 0.2 to 5 mol, preferably from 0.5 to 2 mol, of aniline derivative of the formula (III) are employed per mole of the carbonyl halide of the formula (II). Work-up is carried out by customary methods.

Suitable diluents for carrying out the process (b) according to the invention are all inert organic solvents. These preferably include aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as diethyl ether, diisopropyl ether, methyl tert-butyl ether, methyl tert-amyl ether, dioxane, tetrahydrofuran, 1,2-dimethoxyethane, 1,2-diethoxyethane or anisole, or amides, such as N,N-dimethylformamide, N,N-dimethylacetamide, N-methylformanilide, N-methylpyrrolidone or hexamethylphosphoric triamide.

The process (b) according to the invention is carried out in the presence of a base. Suitable bases are all customary inorganic or organic bases. These preferably include alkaline earth metal or alkali metal hydrides, hydroxides, amides, alkoxides, acetates, carbonates or bicarbonates, such as, for example, sodium hydride, sodium amide, sodium methoxide, sodium ethoxide, potassium tert-butoxide, sodium hydroxide, potassium hydroxide, ammonium hydroxide, sodium acetate, potassium acetate, calcium acetate, ammonium acetate, sodium carbonate, potassium carbonate, potassium bicarbonate, sodium bicarbonate or caesium carbonate, and also tertiary amines, such as trimethylamine, triethylamine, tributylamine, N,N-dimethylaniline, N,N-dimethylbenzylamine, pyridine, N-methylpiperidine, N-methylmorpholine, N,N-dimethyl aminopyridine, diazabicyclooctane (DABCO), diazabicyclononene (DBN) or diazabicycloundecene (DBU).

When carrying out the process (b) according to the invention, the reaction temperatures can be varied within a relatively wide range. In general, the process is carried out at temperatures of from 0° C. to 150° C., preferably at temperatures of from 20° C. to 110° C.

For carrying out the process (b) according to the invention for preparing the compounds of the formula (I), in general from 0.2 to 5 mol, preferably from 0.5 to 2 mol, of halide of the formula (IV) are employed per mole of the thienylpyridylcarboxamide of the formula (I-a).

In general, the processes (a) and (b) according to the invention are carried out under atmospheric pressure. However, it is also possible to operate under elevated or reduced pressure—in general between 0.1 bar and 10 bar.

The compounds according to the invention exhibit a potent microbicidal activity and can be employed in crop protection and in the protection of materials for controlling undesirable microorganisms such as fungi and bacteria.

Fungicides can be employed in crop protection for controlling Plasmodiophoromycetes, Oomycetes, Chytridiomycetes, Zygomycetes, Ascomycetes, Basidiomycetes and Deuteromycetes.

Bactericides can be employed in crop protection for controlling Pseudomonadaceae, Rhizobiaceae, Enterobacteriaceae, Corynebacteriaceae and Streptomycetaceae.

Examples which may be mentioned, but not by limitation, of some pathogens of fungal and bacterial diseases which come under the abovementioned general terms are:

diseases caused by powdery mildew pathogens, such as, for example

Blumeria species such as, for example, Blumeria graminis;

Podosphaera species such as, for example, Podosphaera leucotricha;

Sphaerotheca species such as, for example, Sphaerotheca fuliginea;

Uncinula species such as, for example, Uncinula necator;

diseases caused by rust pathogens such as, for example,

Gymnosporangium species such as, for example, Gymnosporangium sabinae

Hemileia species such as, for example, Hemileia vastatrix;

Phakopsora species such as, for example, Phakopsora pachyrhizi and Phakopsora meibomiae;

Puccinia species such as, for example, Puccinia recondite or Puccinia graminis;

Uromyces species such as, for example, Uromyces appendiculatus;

diseases caused by pathogens from the Oomycetes group such as, for example,

Bremia species such as, for example, Bremia lactucae;

Peronospora species such as, for example, Peronospora pisi or P. brassicae;

Phytophthora species such as, for example, Phytophthora infestans;

Plasmopara species such as, for example, Plasmopara viticola;

Pseudoperonospora species such as, for example, Pseudoperonospora humuli or Pseudoperonospora cubensis;

Pythium species such as, for example, Pythium ultimum;

leaf spot diseases and leaf wilts caused by, for example,

Alternaria species such as, for example, Alternaria solani;

Cercospora species such as, for example, Cercospora beticola;

Cladosporum species such as, for example, Cladosporium cucumerinum;

Cochliobolus species such as, for example, Cochliobolus sativus

(conidial form: Drechslera, syn: Helminthosporium);

Colletotrichum species such as, for example, Colletotrichum lindemuthanium;

Cycloconium species such as, for example, Cycloconium oleaginum;

Diaporthe species such as, for example, Diaporthe citri;

Elsinoe species such as, for example, Elsinoe fawcettii;

Gloeosporium species such as, for example, Gloeosporium laeticolor;

Glomerella species such as, for example, Glomerella cingulata;

Guignardia species such as, for example, Guignardia bidwelli;

Leptosphaeria species such as, for example, Leptosphaeria maculans;

Magnaporthe species such as, for example, Magnaporthe grisea;

Mycosphaerella species such as, for example, Mycosphaerella graminicola and Mycosphaerella fijiensis;

Phaeosphaeria species such as, for example, Phaeosphaeria nodorum;

Pyrenophora species such as, for example, Pyrenophora teres;

Ramularia species such as, for example, Ramularia collo-cygni;

Rhynchosporium species such as, for example, Rhynchosporium secalis;

Septoria species such as, for example, Septoria apii;

Typhula species such as, for example, Typhula incarnata;

Venturia species such as, for example, Venturia inaequalis;

root and stem diseases caused by, for example,

Corticium species such as, for example, Corticium graminearum;

Fusarium species such as, for example, Fusarium oxysporum;

Gaeumannomyces species such as, for example, Gaeumannomyces graminis;

Rhizoctonia species such as, for example, Rhizoctonia solani;

Tapesia species such as, for example, Tapesia acuformis or Tapesia yallundae;

Thielaviopsis species such as, for example, Thielaviopsis basicola;

ear and panicle diseases (including maize cobs), caused by, for example,

Alternaria species such as, for example, Alternaria spp.;

Aspergillus species such as, for example, Aspergillus flavus;

Cladosporium species such as, for example, Cladosporium cladosporioides;

Claviceps species such as, for example, Claviceps purpurea;

Fusarium species such as, for example, Fusarium culmorum;

Gibberella species such as, for example, Gibberella zeae;

Monographella species such as, for example, Monographella nivalis;

diseases caused by smuts such as, for example,

Sphacelotheca species such as, for example, Sphacelotheca reiliana;

Tilletia species such as, for example, Tilletia caries;

Urocystis species such as, for example, Urocystis occulta;

Ustilago species such as, for example, Ustilago nuda;

fruit rots caused by, for example,

Aspergillus species such as, for example, Aspergillus flavus;

Botrytis species such as, for example, Botrytis cinerea;

Penicillium species such as, for example, Penicillium expansum and Penicillium purpurogenum;

Sclerotinia species such as, for example, Sclerotinia sclerotiorum;

Verticilium species such as, for example, Verticilium alboatrum;

seed- and soil-borne rots and wilts, and seedling diseases, caused by, for example,

Fusarium species such as, for example, Fusarium culmorum;

Phytophthora species such as, for example, Phytophthora cactorum;

Pythium species such as, for example, Pythium ultimum;

Rhizoctonia species such as, for example, Rhizoctonia solani;

Sclerotium species such as, for example, Sclerotium rolfsii;

cankers, galls and witches' broom disease, caused by, for example,

Nectria species such as, for example, Nectria galligena;

wilts caused by, for example,

Monilinia species such as, for example, Monilinia laxa;

deformations of leaves, flowers and fruits, caused by, for example,

Taphrina species such as, for example, Taphrina deformans;

degenerative diseases of woody species, caused by, for example,

Esca species such as, for example, Phaeomoniella clamydospora and Phaeoacremonium aleophilum and Fomitiporia mediterranea;

diseases of flowers and seeds, caused by, for example,

Botrytis species such as, for example, Botrytis cinerea;

diseases of the plant tubers, caused by, for example,

Rhizoctonia species such as, for example, Rhizoctonia solani;

Helminthosporium species, such as, for example, Helminthosporium solani;

diseases caused by bacterial pathogens such as, for example,

Xanthomonas species such as, for example, Xanthomonas campestris pv. oryzae;

Pseudomonas species such as, for example, Pseudomonas syringae pv. lachrymans;

Erwinia species such as, for example, Erwinia amylovora;

The following diseases of soybeans can preferably be controlled:

Fungal diseases on leaves, stems, pods and seeds caused by, for example, alternaria leaf spot (Alternaria spec. atrans tenuissima), anthracnose (Colletotrichum gloeosporoides dematium var. truncatum), brown spot (Septoria glycines), cercospora leaf spot and blight (Cercospora kikuchii), choanephora leaf blight (Choanephora infundibulifera trispora (syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi), scab (Sphaceloma glycines), stemphylium leaf blight (Stemphylium botryosum), target spot (Corynespora cassiicola) fungal diseases on roots and the stem base caused by, for example black root rot (Calonectria crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmospora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidermatum, Pythium irregulare, Pythium debaryanum, Pythium myriotylum, Pythium ultimum), rhizoctonia root rot, stem decay, and damping-off (Rhizoctonia solani), sclerotinia stem decay (Sclerotinia sclerotiorum), sclerotinia southern blight (Sclerotinia rolfsii), thielaviopsis root rot (Thielaviopsis basicola).

The active compounds according to the invention also have a potent strengthening effect in plants. They are therefore suitable for mobilizing the plants' defences against attack by undesired microorganisms.

Plant-strengthening (resistance-inducing) substances are understood as meaning, in the present context, those substances which are capable of stimulating the defence system of plants in such a way that, when subsequently inoculated with undesired microorganisms, the treated plants display a substantial degree of resistance to these microorganisms.

In the present case, undesired microorganisms are understood as meaning phytopathogenic fungi, bacteria and viruses. Thus, the substances according to the invention can be employed for protecting plants against attack by the abovementioned pathogens within a certain period of time after the treatment. The period of time within which their protection is effected is generally extended from 1 to 28 days, preferably 1 to 14 days, particularly preferably 1 to 7 days, after the plants have been treated with the active compounds.

The fact that the active compounds, at the concentrations required for the controlling of plant diseases, are well tolerated by plants permits the treatment of aerial plant parts, of vegetative propagation material and seed, and of the soil.

In this context, the active compounds according to the invention can be employed particularly successfully for controlling cereal diseases such as, for example, against Puccinia species and diseases in viticulture, fruit production and vegetable production such as, for example against Botrytis, Venturia or Alternaria species.

The active compounds according to the invention are also suitable for increasing the yield. Moreover, they display a low degree of toxicity and are well tolerated by plants.

If appropriate, the active compounds according to the invention can also be used in certain concentrations and application rates as herbicides, for influencing plant growth and for controlling animal pests. If appropriate, they can also be employed as intermediates and precursors for the synthesis of further active compounds.

All plants and plant parts can be treated in accordance with the invention. Plants are understood as meaning, in the present context, all plants and plant populations, such as desired and undesired wild plants or crop plants (including naturally occurring crop plants). Crop plants may be plants which can be obtained by conventional breeding and optimization methods or else by biotechnological and genetic engineering methods or by combinations of these methods, including the transgenic plants and including the plant varieties capable or not capable of being protected by Plant Breeders' rights. Plant parts are understood as meaning all aerial and subterranean parts and organs of the plants, such as shoot, leaf, flower and root, examples which may be mentioned being leaves, needles, stalks, stems, flowers, fruiting bodies, fruits and seeds, and also roots, tubers and rhizomes. The plant parts also include harvested material and vegetative and generative propagation material, for example cuttings, tubers, rhizomes, slips and seeds.

The treatment according to the invention with the active compounds, of the plants and plant parts, is carried out directly or by acting on their environment, habitat, or store by the customary treatment methods, for example by immersion, spraying, vaporizing, fogging, broadcasting, painting on and, in the case of propagation material, in particular in the case of seeds, furthermore by coating with one or more coats.

In the protection of materials, the substances according to the invention can be employed for protecting industrial materials against attack and destruction by undesired microorganisms.

In the present context, industrial materials are understood as meaning non live materials which have been made for use in technology. For example, industrial materials which are to be protected by active compounds according to the invention from microbial modification or destruction can be glues, sizes, paper and board, textiles, leather, timber, paints and plastic articles, cooling lubricants and other materials which are capable of being attacked or destroyed by microorganisms. Parts of production plants, for example cooling-water circuits, which can be adversely affected by the multiplication of microorganisms may also be mentioned within the materials to be protected. Industrial materials which may be mentioned with preference for the purposes of the present invention are glues, sizes, paper and board, leather, timber, paints, cooling lubricants and heat-transfer fluids, especially preferably wood.

Microorganisms which are capable of bringing about a degradation or modification of the industrial materials and which may be mentioned are, for example, bacteria, fungi, yeasts, algae and slime organisms. The active compounds according to the invention are preferably active against fungi, in particular moulds, wood-discolouring and wood-destroying fungi (Basidiomycetes) and against slime organisms and algae.

Examples which may be mentioned are microorganisms of the following genera:

Alternaria such as Alternaria tenuis,

Aspergillus such as Aspergillus niger,

Chaetomium such as Chaetomium globosum,

Coniophora such as Coniophora puetana,

Lentinus such as Lentinus tigrinus,

Penicillium such as Penicillium glaucum,

Polyporus such as Polyporus versicolor,

Aureobasidium such as Aureobasidium pullulans,

Sclerophoma such as Sclerophoma pityophila,

Trichoderma such as Trichoderma viride,

Escherichia such as Escherichia coli,

Pseudomonas such as Pseudomonas aeruginosa,

Staphylococcus such as Staphylococcus aureus.

Depending on their respective physical and/or chemical properties, the active compounds can be converted to the customary formulations, such as solutions, emulsions, suspensions, powders, foams, pastes, granules, aerosols, very fine capsules in polymeric substances and in coating compositions for seed, and also ULV cold- and warm-fogging formulations.

These formulations are produced in a known manner, for example by mixing the active compounds with extenders, that is, liquid solvents, pressurized liquefied gases and/or solid carriers, optionally with the use of surface-active agents, that is emulsifers and/or dispersants, and/or foam formers. If the extender used is water, it is also possible to employ for example organic solvents as cosolvents. Suitable liquid solvents are essentially: aromatics, such as xylene, toluene or alkylnaphthalenes, chlorinated aromatics or chlorinated aliphatic hydrocarbons, such as chlorobenzenes, chloroethylenes or methylene chloride, aliphatic hydrocarbons, such as cyclohexane or paraffins, for example mineral oil fractions, alcohols, such as butanol or glycol as well as their ethers and esters, ketones, such as acetone, methyl ethyl ketone, methyl isobutyl ketone or cyclohexanone, strongly polar solvents, such as dimethylformamide and dimethyl sulphoxide, and also water. Liquefied gaseous extenders or carriers are those liquids which are gaseous at ambient temperature and at atmospheric pressure, for example aerosol propellants such as halogenated hydrocarbons and also butane, propane, nitrogen and carbon dioxide. As solid carriers there are suitable: for example ground natural minerals, such as kaolins, clays, talc, chalk, quartz, attapulgite, montmorillonite or diatomaceous earth, and ground synthetic minerals, such as finely divided silica, alumina and silicates. As solid carriers for granules there are suitable: for example crushed and fractionated natural rocks such as calcite, pumice, marble, sepiolite and dolomite, and also synthetic granules of inorganic and organic meals, and granules of organic material such as sawdust, coconut shells, maize cobs and tobacco stalks. As emulsifiers and/or foam formers there are suitable: for example non-ionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and protein hydrolysates. As dispersants there are suitable: for example lignosulphite waste liquors and methylcellulose.

Tackifiers such as carboxymethylcellulose and natural and synthetic polymers in the form of powders, granules or latices, such as gum arabic, polyvinyl alcohol and polyvinyl acetate, as well as natural phospholipids, such as cephalins and lecithins, and synthetic phospholipids, can be used in the formulations. Other possible additives are mineral and vegetable oils.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

The formulations in general contain between 0.1 and 95% by weight of active compound, preferably between 0.5 and 90%.

The active compounds according to the invention, as such or in their formulations, can also be used as a mixture with known fungicides, bactericides, acaricides, nematicides, or insecticides, for example, to improve the activity spectrum or prevent the development of resistance. In many instances, synergistic effects are obtained, i.e. the activity of the mixture exceeds the activity of the individual components.

Examples of co-components in mixtures are the following compounds:

Fungicides:

1) Nucleic acid synthesis inhibitors: for example benalaxyl, benalaxyl-M, bupirimate, clozylacon, dimethirimol, ethirimol, furalaxyl, hymexazol, mefenoxam, metalaxyl, metalaxyl-M, ofurace, oxadixyl, oxolinic acid;

2) mitosis and cell division inhibitors: for example benomyl, carbendazim, diethofencarb, ethaboxam, fuberidazole, pencycuron, thiabendazole, thiophanate-methyl, zoxamide;

3) respiration inhibitors (inhibitors of the respiratory chain):

3.1) inhibitors which act on complex I of the respiratory chain: for example diflumetorim;

3.2) inhibitors which act on complex II of the respiratory chain: for example boscalid/nicobifen, carboxin, fenfuram, flutolanil, furametpyr, furmecyclox, mepronil, oxycarboxin, penthiopyrad, thifluzamide;

3.3) inhibitors which act on complex III of the respiratory chain: for example amisulbrom, azoxystrobin, cyazofamid, dimoxystrobin, enestrobin, famoxadone, fenamidone, fluoxastrobin, kresoxim-methyl, meto-minostrobin, orysastrobin, picoxystrobin, pyraclostrobin, trifloxystrobin;

4) decouplers: for example dinocap, fluazinam, meptyldinocap;

5) ATP production inhibitors: for example fentin acetate, fentin chloride, fentin hydroxide, silthiofam;

6) amino acid and protein biosynthesis inhibitors: for example andoprim, blasticidin-S, cyprodinil, kasugamycin, kasugamycin hydrochloride hydrate, mepanipyrim, pyrimethanil;

7) signal transduction inhibitors: for example fenpiclonil, fludioxonil, quinoxyfen;

8) lipid and membrane synthesis inhibitors: for example biphenyl, chlozolinate, edifenphos, iodocarb, iprobenfos, iprodione, isoprothiolane, procymidone, propamocarb, propamocarb hydrochloride, pyrazophos, tolclofos-methyl, vinclozolin;

9) ergosterol biosynthesis inhibitors: for example aldimorph, azaconazole, bitertanol, bromuconazole, cyproconazole, diclobutrazole, difenoconazole, diniconazole, diniconazole-M, dodemorph, dodemorph acetate, epoxiconazole, etaconazole, fenarimol, fenbuconazole, fenhexamid, fenpropidin, fenpropimorph, fluquinconazole, flurprimidol, flusilazole, flutriafol, furconazole, furconazole-cis, hexaconazole, imazalil, imazalil sulfate, imibenconazole, ipconazole, metconazole, myclobutanil, naftifine, nuarimol, oxpoconazole, paclobutrazol, pefurazoate, penconazole, prochloraz, propiconazole, prothioconazole, pyributicarb, pyrifenox, simeconazole, spiroxamine, tebuconazole, terbinafine, tetraconazole, triadimefon, triadimenol, tridemorph, triflumizole, triforine, triticonazole, uniconazole, viniconazole, voriconazole;

10) cell wall synthesis inhibitors: for example benthiavalicarb, dimethomorph, flumorph, iprovalicarb, polyoxins, polyoxorim, validamycin A;

11) melanin biosynthesis inhibitors: for example carpropamid, diclocymet, fenoxanil, phthalide, pyroquilon, tricyclazole;

12) resistance inductors: for example acibenzolar-S-methyl, probenazole, tiadinil;

13) compounds with multi-site activity: for example Bordeaux mixture, captafol, captan, chlorothalonil, copper naphthenate, copper oxide, copper oxychloride, copper preparations such as, for example, copper hydroxide, copper sulphate, dichlofluanid, dithianon, dodine, dodine free base, ferbam, fluorofolpet, folpet, guazatine, guazatine acetate, iminoctadine, iminoctadine albesilate, iminoctadine triacetate, mancopper, mancozeb, maneb, metiram, metiram zinc, oxine-copper, propineb, sulphur and sulphur preparations such as, for example, calcium polysulphide, thiram, tolylfluanid, zineb, ziram;

14) a compound selected from the following enumeration: N-methyl-(2E)-2-(2-{[6-(3-chloro-2-methylphenoxy)-5-fluoropyrimidin-4-yl]oxy}phenyl)-2-(methoxyimino)acetamide, N-methyl-(2E)-2-{2-[({[(1E)-1-(3-{[(E)-1-fluoro-2-phenylvinyl]oxy}phenyl)ethylidene]amino}oxy)methyl]phenyl}-2-(methoxyimino)acetamide, 1-(4-chlorophenyl)-2-(1H-1,2,4-triazol-1-yl)cycloheptanol, 1-[(4-methoxyphenoxy)methyl]-2,2-dimethylpropyl-1H-imidazole-1-carboxylate, 2-(4-chlorophenyl)-N-{2-[3-methoxy-4-(prop-2-yn-1-yloxy)phenyl]ethyl}-2-(prop-2-yn-1-yloxy)acetamide, 2,3,5,6-tetrachloro-4-(methylsulphonyl)pyridine, 2-butoxy-6-iodo-3-propyl-4H-chromen-4-one, 2-chloro-N-(1,1,3-trimethyl-2,3-dihydro-1H-inden-4-yl)nicotinamide, 2-phenylphenol and salts thereof, 3,4,5-trichloropyridine-2,6-dicarbonitrile, 3,4-dichloro-N-(2-cyanophenyl)isothiazole-5-carboxamide, 3-[5-(4-chlorophenyl)-2,3-dimethylisoxazolidin-3-yl]pyridine, 5-chloro-6-(2,4,6-trifluorophenyl)-N-[(1R)-1,2,2-trimethylpropyl][1,2,4]triazolo[1,5-a]pyrimidine-7-amine, 5-chloro-7-(4-methylpiperidin-1-yl)-6-(2,4,6-trifluorophenyl)[1,2,4]triazolo[1,5-a]pyrimidine, 5-chloro-N-[(1R)-1,2-dimethylpropyl]-6-(2,4,6-trifluorophenyl)[1,2,4]triazolo[1,5-a]pyrimidine-7-amine, 8-hydroxyquinoline sulphate, benthiazole, bethoxazin, capsimycin, carvone, quinomethionate, cufraneb, cyflufenamid, cymoxanil, dazomet, debacarb, dichlorophen, diclomezine, dicloran, difenzoquat, difenzoquat methylsulphate, diphenylamine, ferimzone, flumetover, fluopicolide, fluoroimide, flusulfamide, fosetyl-aluminium, fosetyl-calcium, fosetyl-sodium, hexachlorobenzene, irumamycin, methasulfocarb, methyl(2-chloro-5-{(1E)-N-[(6-methylpyridin-2-yl)methoxy]ethanimidoyl}benzyl)carbamate, methyl(2E)-2-{2-[({cyclopropyl[(4-methoxyphenyl)imino]methyl}thio)methyl]phenyl}-3-methoxyacrylate, methyl 1-(2,2-dimethyl-2,3-dihydro-1H-inden-1-yl)-1H-imidazole-5-carboxylate, methyl 3-(4-chlorophenyl)-3-{[N-(isopropoxycarbonyl)valyl]amino}propanoate, methyl isothiocyanate, metrafenone, mildiomycin, N-(3′,4′-dichloro-5-fluorobiphenyl-2-yl)-3-(difluoromethyl)-1-methyl-1H-pyrazole-4-carboxamide, N-(3-ethyl-3,5,5-trimethylcyclohexyl)-3-(formylamino)-2-hydroxybenzamide, N-(4-chloro-2-nitrophenyl)-N-ethyl-4-methylbenzenesulphonamide, N-[(5-bromo-3-chloropyridin-2-yl)methyl]-2,4-dichloronicotinamide, N-[1-(5-bromo-3-chloropyridin-2-yl)ethyl]-2,4-dichloronicotinamide, N-[1-(5-bromo-3-chloropyridin-2-yl)ethyl]-2-fluoro-4-iodonicotinamide, N-[2-(4-{[3-(4-chlorophenyl)prop-2-yn-1-yl]oxy}-3-methoxyphenyl)ethyl]-N²-(methylsulphonyl)valinamide, N-{(Z)-[(cyclopropylmethoxy)imino][6-(difluoromethoxy)-2,3-difluorophenyl]methyl}-2-phenylacetamide, N-{2-[3-chloro-5-(trifluoromethyl)pyridin-2-yl]ethyl}-2-(trifluoromethyl)benzamide, natamycin, nickel dimethyl dithiocarbamate, nitrothal-isopropyl, O-{1-[(4-methoxyphenoxy)methyl]-2,2-dimethylpropyl}1H-imidazole-1-carbothioate, octhilinone, oxamocarb, oxyfenthiin, pentachlorophenol and salts, phosphoric acid and its salts, piperalin, propamocarb fosetylate, propanosine-sodium, proquinazid, pyrrolnitrin, quintozene, tecloftalam, tecnazene, triazoxide, trichlamide, zarilamid.

Bactericides:

Bronopol, dichlorophen, nitrapyrin, nickel dimethyl dithiocarbamate, kasugamycin, octhilinone, furan-carboxylic acid, oxytetracyclin, probenazole, streptomycin, tecloftalam, copper sulphate and other copper preparations.

Insecticides/Acaricides/Nematicides:

1. Acetylcholine esterase (AChE) inhibitors

1.1 Carbamates (for example alanycarb, aldicarb, aldoxycarb, allyxycarb, aminocarb, azamethiphos, bendiocarb, benfuracarb, bufencarb, butacarb, butocarboxim, butoxycarboxim, carbaryl, carbofuran, carbosulfan, chloethocarb, coumaphos, cyanofenphos, cyanophos, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, triazamate, trimethacarb, XMC, xylylcarb)

1.2 Organophosphates (for example acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-S-methyl, demeton-S-methylsulphon, dialifos, diazinon, dichlofenthion, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion)

2. Sodium channel modulators/voltage-dependent sodium channel blockers

2.1 Pyrethroids (for example acrinathrin, allethrin (d-cis-trans, d-trans), beta-cyfluthrin, bifenthrin, bio-allethrin, bioallethrin-S-cyclopentyl isomer, bioethanomethrin, biopermethrin, bioresmethrin, chlo-vaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, DDT, deltamethrin, empenthrin (1R isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R trans-isomer), prallethrin, profluthrin, protrifenbute, pyresmethrin, resmethrin, RU 15525, silafluofen, tau-fluvalinate, tefluthrin, terallethrin, tetramethrin (1R isomer), tralomethrin, transfluthrin, ZXI 8901, pyrethrins (pyrethrum))

2.2 Oxadiazines (for example indoxacarb)

3. Acetylcholine receptor agonists/antagonists

3.1 Chloronicotinyls/neonicotinoids (for example acetamiprid, clothianidin, dinotefuran, imidacloprid, nitenpyram, nithiazine, thiacloprid, thiamethoxam)

3.2 Nicotine, bensultap, cartap

4. Acetylcholine receptor modulators

4.1 Spinosyns (for example spinosad)

5. GABA-controlled chloride channel antagonists

5.1 Cyclodiene organochlorines (for example camphechlor, chlordane, endosulfan, gamma-HCH, HCH, heptachlor, lindane, methoxychlor

5.2 Fiproles (for example acetoprole, ethiprole, fipronil, vaniliprole)

6. Chloride channel activators

6.1 Mectins (for example abamectin, avermectin, emamectin, emamectin benzoate, ivermectin, milbemectin, milbemycin)

7. Juvenile hormone mimetics

(for example diofenolan, epofenonane, fenoxycarb, hydroprene, kinoprene, methoprene, pyriproxifen, triprene)

8. Ecdysone agonists/disruptors

8.1 Diacylhydrazines (for example chromafenozide, halofenozide, methoxyfenozide, tebufenozide)

9. Chitin biosynthesis inhibitors

9.1 Benzoylureas (for example bistrifluron, chlofluazuron, diflubenzuron, fluazuron, flucycloxuron, flufenoxuron, hexaflumuron, lufenuron, novaluron, noviflumuron, penfluron, teflubenzuron, triflumuron)

9.2 Buprofezin

9.3 Cyromazine

10. Inhibitors of oxidative phosphorylation, ATP disruptors

10.1 Diafenthiuron

10.2 Organotins (for example azocyclotin, cyhexatin, fenbutatin oxide)

11. Uncouplers of oxidative phosphorylation by interrupting the H-proton gradient

11.1 Pyrroles (for example chlorfenapyr)

11.2 Dinitrophenols (for example binapacyrl, dinobuton, dinocap, DNOC)

12. Site-I electron transport inhibitors

12.1 METIs (for example fenazaquin, fenpyroximate, pyrimidifen, pyridaben, tebufenpyrad, tolfenpyrad)

12.2 Hydramethylnone

12.3 Dicofol

13. Site-II electron transport inhibitors

13.1 Rotenone

14. Site-Ill electron transport inhibitors

14.1 Acequinocyl, fluacrypyrim

15. Microbial disruptors of the insect gut membrane

Bacillus thuringiensis strains

16. Fat synthesis inhibitors

16.1 Tetronic acids (for example spirodiclofen, spiromesifen)

16.2 Tetramic acids [for example 3-(2,5-dimethylphenyl)-8-methoxy-2-oxo-1-azaspiro[4.5]dec-3-en-4-yl ethyl carbonate (also known as: carbonic acid, 3-(2,5-dimethylphenyl)-8-methoxy-2-oxo-1-azaspiro[4.5]dec-3-en-4-yl ethyl ester, CAS Reg. No.: 382608-10-8) and carbonic acid, cis-3-(2,5-dimethylphenyl)-8-methoxy-2-oxo-1-azaspiro[4.5]dec-3-en-4-yl ethyl ester (CAS Reg.-No.: 203313-25-1)]

17. Carboxamides

(for example flonicamid)

18. Octopaminergic agonists

(for example amitraz)

19. Magnesium-stimulated ATPase inhibitors

(for example propargite)

20. Ryanodin receptor agonists

20.1 Benzoic acid dicarboxamides (for example N²-[1,1-dimethyl-2-(methylsulfonyl)ethyl]-3-iodo-N¹-[2-methyl-4-[1,2,2,2-tetrafluoro-1-(trifluoromethyl)ethyl]phenyl]-1,2-benzenedicarboxamide (CAS Reg.-No.: 272451-65-7), flubendiamide)

20.2 Anthranilamide (for example DPX E2Y45=3-bromo-N-{4-chloro-2-methyl-6-[(methylamino)carbonyl]-phenyl}-1-(3-chloropyridin-2-yl)-1H-pyrazol-5-carboxamide)

21. Nereistoxin analogues

(for example thiocyclam hydrogen oxalate, thiosultap sodium)

22. Biologicals, hormones or pheromones

(for example azadirachtin, Bacillus spec., Beauveria spec., codlemone, Metarrhizium spec., Paecilomyces spec., thuringiensin, Verticillium spec.)

23. Active compounds with unknown or unspecific mechanisms of action

23.1 Fumigants (for example aluminium phosphide, methyl bromide, sulphuryl fluoride)

23.2 Selective antifeedants (for example cryolite, flonicamid, pymetrozine)

23.3 Mite growth inhibitors (for example clofentezine, etoxazole, hexythiazox)

23.4 Amidoflumet, benclothiaz, benzoximate, bifenazate, bromopropylate, buprofezin, quinomethionate, chlordimeform, chlorobenzilate, chloropicrin, clothiazoben, cycloprene, cyflumetofen, dicyclanil, fenoxacrim, fentrifanil, flubenzimine, flufenerim, flutenzin, gossyplure, hydramethylnone, japonilure, metoxadiazone, petroleum, piperonyl butoxide, potassium oleate, pyrafluprole, pyridalyl, pyriprole, sulfluramid, tetradifon, tetrasul, triarathene, verbutin, furthermore the compound 3-methyl-phenyl propylcarbamate (tsumacide Z), the compound 3-(5-chloro-3-pyridinyl)-8-(2,2,2-trifluoroethyl)-8-azabicyclo[3.2.1]octane-3-carbonitrile (CAS Reg. No. 185982-80-3) and the corresponding 3-endo isomer (CAS Reg. No. 185984-60-5) (cf. WO 96/37494, WO 98/25923), and preparations which contain insecticidally active plant extracts, nematodes, fungi or viruses.

A mixture with other known active compounds such as herbicides, or with fertilizers and growth regulators, safeners or semiochemicals is also possible.

In addition, the compounds of the formula (I) according to the invention also have very good antimycotic activity. They have a very broad antimycotic spectrum of action, in particular against dermatophytes and budding fungi, moulds and diphasic fungi (for example against Candida species such as Candida albicans, Candida glabrata) and Epidermophyton floccosum, Aspergillus species such as Aspergillus niger and Aspergillus fumigatus, Trichophyton species such as Trichophyton mentagrophytes, Microsporon species such as Microsporon canis and audouinii. The enumeration of these fungi is no restriction whatsoever of the mycotic spectrum which can be controlled and is provided by illustration only.

The active compounds can be employed as such, in the form of their formulations or the use forms prepared therefrom, such as ready-to-use solutions, suspensions, wettable powders, pastes, soluble powders, dusts and granules. They are applied in the customary manner, for example by pouring, spraying, atomizing, broadcasting, dusting, foaming, painting on and the like. It is furthermore possible to apply the active compounds by the ultra-low-volume method, or to inject the active compound preparation or the active compound itself into the soil. The seed of the plant can also be treated.

When employing the active compounds according to the invention as fungicides, the application rates can be varied within a substantial range, depending on the type of application. In the treatment of plant parts, the application rates of active compound are generally between 0.1 and 10 000 g/ha, preferably between 10 and 1000 g/ha. For the treatment of seed, the application rates of active compound are generally between 0.001 and 50 g per kilogram of seed, preferably between 0.01 and 10 g per kilogram of seed. For treating the soil, the application rates of active compound are generally between 0.1 and 10 000 g/ha, preferably between 1 and 5000 g/ha.

As already mentioned above, all plants and their parts can be treated in accordance with the invention. In a preferred embodiment, plant species and plant varieties which are found in the wild or are obtained by traditional biological breeding methods, such as hybridization or protoplast fusion, and parts of the former are treated. In a further preferred embodiment, transgenic plants and plant varieties which have been obtained by recombinant methods, if appropriate in combination with traditional methods (genetically modified organisms) and their parts are treated. The term “parts” or “parts of plants” or “plant parts” has been illustrated above.

Particularly preferably, plants of the plant cultivars which are in each case commercially available or in use are treated according to the invention. Plant cultivars are understood as meaning plants with new properties (“traits”) which have been obtained by conventional cultivation, by mutagenesis or else by recombinant DNA techniques. These may be cultivars, breeds, biotypes or genotypes.

Depending on the plant species or plant cultivars, their location and growth conditions (soils, climate, vegetation period, nutrition), the treatment according to the invention may also result in superadditive (“synergistic”) effects. Thus, for example, reduced application rates and/or extensions of the activity spectrum and/or an increase in the activity of the substances and compositions that can be used according to the invention, better plant growth, more developed root system, higher resistance of the plant variety or plant cultivar, increased growth of shoots, higher plant vitality, increased tolerance to high or low temperatures, increased tolerance to drought or to water or soil salinity, increased flowering performance, easier harvesting, accelerated maturation, higher harvest yields, larger fruit, increased plant size, greener leaf colour, earlier blossoming, better quality and/or a higher nutritional value of the harvested products, higher sugar concentration in the fruits, better storage stability and/or processability of the harvested products which exceed the effects which were actually to be expected are possible.

The preferred transgenic plants or plant cultivars (i.e. those obtained by genetic engineering) which are to be treated according to the invention include all plants which, as a result of the recombinant modification, received genetic material which imparted particularly advantageous useful properties (“traits”) to these plants. Examples of such properties are better plant growth, increased tolerance to high or low temperatures, increased tolerance to drought or to water or soil salinity, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, tobacco, oilseed rape and also fruit plants (with the fruits apples, pears, citrus fruits and grapes), and particular emphasis is given to maize, soya beans, potatoes, cotton, tobacco and oilseed rape. Traits that are emphasized in particular are increased defence of the plants against insects, arachnids, nematodes, slugs and snails as the result of toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (hereinbelow referred to as “Bt plants”). Traits which are also particularly emphasized are the increased defence of plants against fungi, bacteria and viruses by systemic acquired resistance (SAR), systemin, phytoalexins, elicitors and resistance genes and the correspondingly expressed proteins and toxins. Traits that are furthermore particularly emphasized are the increased tolerance of the plants to certain herbicidally active compounds, for example imidazolinones, sulphonylureas, glyphosate or phosphinothricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucoton® (cotton) and NewLeaf® (potato). Examples of herbicide-tolerant plants which may be mentioned are maize varieties, cotton varieties and soya bean varieties which are sold under the trade names Roundup Ready® (tolerance to glyphosates, for example maize, cotton, soya bean), Liberty Link® (tolerance to phosphinothricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned also include the varieties sold under the name Clearfield® (for example maize). Of course, these statements also apply to plant cultivars having these genetic traits or genetic traits still to be developed, which cultivars will be developed and/or marketed in the future.

The plants listed can be treated according to the invention in a particularly advantageous manner with the compounds of the general formula (I) or the active compound mixtures according to the invention. The preferred ranges stated above for the active compounds or mixtures also apply to the treatment of these plants. Particular emphasis is given to the treatment of plants with the compounds or mixtures specifically mentioned in the present text.

The preparation and the use of the active compounds according to the invention is illustrated by the examples below.

### PREPARATION EXAMPLES

### Example 1

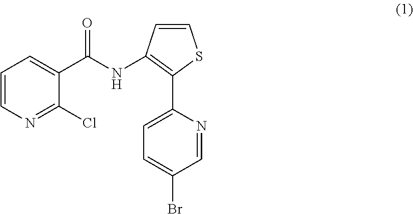

45 mg (0.18 mmol) of 2-(5-bromopyridin-2-yl)thiophene-3-amine are dissolved in 1 ml of acetonitrile, and 27 mg (0.19 mmol) of potassium carbonate are added. This is followed by the addition of 34 mg (0.19 mmol) of 2-chloronicotinoyl chloride. The reaction solution is stirred at room temperature for 3 h. For work-up, the reaction mixture is added to water and extracted with ethyl acetate, and the extracts are concentrated. The crude product is purified by HPLC. This gives 0.12 mg (14% of theory) of N-[2-(5-bromopyridin-2-yl)-3-thienyl]-2-chloronicotinamide of logP (pH 2.3)=2.25.

Preparation of Starting Materials of the Formula (III)

### Example (III-1)

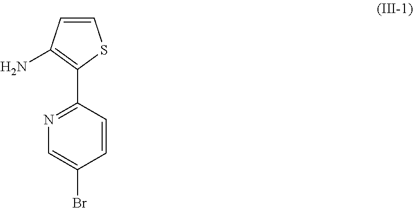

0.1 g (0.28 mmol) of tert-butyl [2-(5-bromopyridin-2-yl)-3-thienyl]carbamate is dissolved in 4.5 ml of dichloromethane and, after addition of 0.7 g (6.1 mmol) of trifluoroacetic acid, stirred at room temperature for 16 h and then at 40° C. for 2 h. For work-up, the mixture is washed twice with 2N aqueous sodium hydroxide solution and once with water, and the organic phase is dried and concentrated. This gives 50 mg of 2-(5-bromopyridin-2-yl)thiophene-3-amine of logP (pH 2.3)=0.87, which was employed directly without further purification.

Preparation of Starting Materials of the Formula (III-a)

### Example (III-a-1)

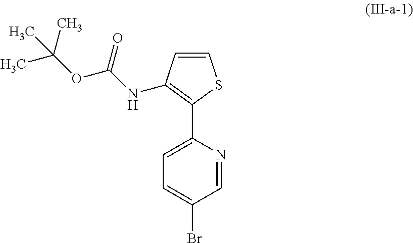

1.6 g (4.6 mmol) of 2-(5-bromopyridin-2-yl)-6-phenyl-1,3,6,2-dioxazaborocane, 1.0 g (3.1 mmol) of tert-butyl-(2-iodo-3-thienyl)carbamate, 162 mg (0.23 mmol) of bis(triphenylphospine)palladium(II) chloride and 2.44 g (23.1 mmol) of sodium carbonate are dissolved in a mixture of 7.5 ml of water and 15 ml of dimethyl sulphoxide. The reaction solution is stirred at 90° C. for 18 h. For work-up, the reaction mixture is added to water and extracted with ethyl acetate, and the extract is concentrated. Column chromatography (cyclohexane/ethyl acetate, gradient) gives 200 mg (12% of theory) of tert-butyl[2-(5-bromopyridin-2-yl)-3-thienyl]carbamate of logP (pH 2.3)=3.27.

Preparation of Starting Materials of the Formula (V)

### Example (V-1)

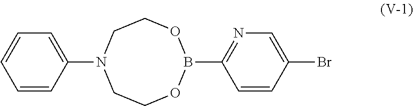

1.0 g (3.5 mmol) of 5-bromo-2-iodopyridine and 0.79 g (4.2 mmol) of triisopropyl borate are dissolved in 13 ml of tetrahydrofuran. The reaction solution is cooled to −75° C., and 271 mg (4.2 mmol) of n-butyllithium are added dropwise. After thawing to room temperature and a further 16 h of stirring, a solution of 638 mg (3.5 mmol) of N-phenyldiethanolamine in a little tetrahydrofuran is added dropwise, and the mixture is then heated under reflux for 4 h. For work-up, the reaction mixture is repeatedly diluted with isopropanol and evaporated to dryness. This gives 1.8 g of 2-(5-bromopyridin-2-yl)-6-phenyl-1,3,6,2-dioxazaborocane, which was used directly without further purification.

Preparation of Starting Materials of the Formula (VI)

### Example (VI-1)

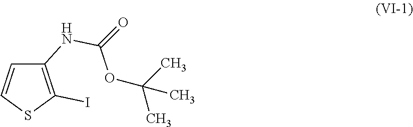

Under protective gas, 4.0 g of tert-butyl 3-thienylcarbamate are dissolved in 30 ml of tetrahydrofuran. The reaction solution is cooled to −78° C., and 3.21 g (50 mmol) of n-buthyllithium are added dropwise. After 30 min of stirring at −78° C., a solution of 6.11 g (24.1 mmol) of iodine in a little tetrahydrofuran is added dropwise, and the mixture is then stirred at −10° C. for 1 h. For work-up, saturated sodium chloride solution is added to the reaction mixture, and the organic phase is evaporated to dryness. Column chromatography (cyclohexane/ethyl acetate 10:1) gives 3.4 g (49% of theory) of tert-butyl (2-iodo-3-thienyl)carbamate of logP (pH 2.3)=2.43.

The logP values given in the Tables and Preparation Examples above are determined in accordance with EEC Directive 79/831 Annex V.A8 by HPLC (High Performance Liquid Chromatography) on a reverse-phase column (C 18). Temperature: 43° C.

The determination is carried out in the acidic range at pH 2.3 using the mobile phases 0.1% aqueous phosphoric acid and acetonitrile; linear gradient from 10% acetonitrile to 90% acetonitrile.

Calibration is carried out using unbranched alkan-2-ones (having 3 to 16 carbon atoms), with known logP values (determination of the logP values by the retention times using linear interpolation between two successive alkanones).

The lambda max values were determined in the maxima of the chromatographic signals using the UV spectra from 200 nm to 400 nm.

1. A thienylpyridylcarboxamide of formula (I) 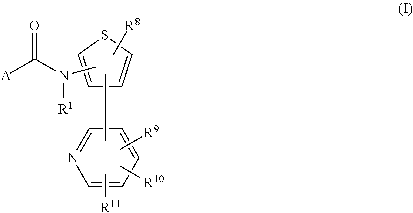
in which
R¹ represents hydrogen, C₁-C₈-alkyl, C₁-C₈-alkylsulphinyl, C₁-C₆-alkylsulphonyl, C₁-C₄-alkoxy-C₁-C₄-alkyl, C₃-C₈-cycloalkyl; C₁-C₆-haloalkyl, C₁-C₄-haloalkylthio, C₁-C₄-haloalkylsulphinyl, C₁-C₄-haloalkylsulphonyl, halo-C₁-C₄-alkoxy-C₁-C₄-alkyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₄alkyl, halo-(C₁-C₃-alkoxy)carbonyl-C₁-C₃-alkyl having in each case 1 to 13 fluorine, chlorine and/or bromine atoms;
(C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₈-alkynyloxy)carbonyl, (C₃-C₈-cycloalkyl)carbonyl; (C₁-C₈-haloalkyl)carbonyl, (C₁-C₆-haloalkoxy)carbonyl, (C₁-C₈-haloalkylthio)carbonyl, (halo-C₁-C₄-alkoxy-C₁-C₄-alkyl)carbonyl, (C₃-C₆-haloalkenyloxy)carbonyl, (C₃-C₆-haloalkynyloxy)carbonyl, (C₃-C₈-halocycloalkyl)carbonyl having in each case 1 to 9 fluorine, chlorine and/or bromine atoms; or —CH₂—C≡C—R^{1-A}, —CH₂—CH═CH—R^{1-A}, —CH═C═CH—R^{1-A}, —C(═O)C(═O)R², —CONR³R⁴ or —CH₂NR⁵R⁶,
R^{1-A} represents hydrogen, C₁-C₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)carbonyl or cyano,
R² represents hydrogen, C₁-0₅-alkyl, C₁-C₈-alkoxy, C₁-C₄-alkoxy-C₁-C₄-alkyl, C₃-C₈-cycloalkyl; C₁-C₆-haloalkyl, C₁-C₆-haloalkoxy, halo-C₁-C₄-alkoxy-C₁-C₄-alkyl, C₃-C₈-halocycloalkyl having in each case 1 to 9 fluorine, chlorine and/or bromine atoms,
R³ and R⁴ independently of one another each represent hydrogen, C₁-C₈-alkyl, C₁-C₄-alkoxy-C₁-C₄-alkyl, C₃-C₈-cycloalkyl; C₁-C₈-haloalkyl, halo-C₁-C₄-alkoxy-C₁-C₄-alkyl, C₃-C₈-halocycloalkyl having in each case 1 to 9 fluorine, chlorine and/or bromine atoms,
R³ and R⁴ furthermore together with the nitrogen atom to which they are attached form a saturated heterocycle which is mono- or polysubstituted by identical or different substituents from the group consisting of halogen and C₁-C₄-alkyl and which has 5 to 8 ring atoms, where the heterocycle may contain 1 or 2 further non-adjacent heteroatoms from the group consisting of oxygen, sulphur and NR⁷,
R⁵ and R⁶ independently of one another each represent hydrogen, C₁-C₈-alkyl, C₃-C₈-cycloalkyl; C₁-C₈-haloalkyl, C₃-C₈-halocycloalkyl having in each case 1 to 9 fluorine, chlorine and/or bromine atoms,
R⁵ and R⁶ furthermore together with the nitrogen atom to which they are attached form a saturated heterocycle which is mono- or polysubstituted by identical or different substituents from the group consisting of halogen and C₁-C₄-alkyl and which has 5 to 8 ring atoms, where the heterocycle may contain 1 or 2 further non-adjacent heteroatoms from the group consisting of oxygen, sulphur and NR⁷,
R⁷ represents hydrogen or C₁-C₆-alkyl,
R⁸ represents hydrogen, fluorine, chlorine, methyl, isopropyl, methylthio or trifluoromethyl,
R⁹, R¹⁰ and R¹¹ independently of one another represent hydrogen, halogen, cyano, nitro, C₁-C₆-alkyl, C₂-C₆-alkenyl, C₂-C₆-alkynyl, C₁-C₄-alkoxy, C₁-C₄-alkylsulphinyl, C₁-C₄-alkylsulphonyl, C₃-C₆-cycloalkyl; represent C₁C₄-haloalkyl, C₁-C₄-haloalkoxy, C₁-C₄-haloalkylthio, C₁-C₄-haloalkylsulphinyl, C₁-C₄-haloalkylsulphonyl having in each case 1 to 5 halogen atoms; represent —SO₂NR¹²R¹³, —C(═X)R¹⁴, —NR¹²R¹³, —CH₂—NR¹²R¹³,
R¹² represents hydrogen, C₁-C₄-alkyl or —C(═X)R¹⁴,
R¹³ represents hydrogen, C₁-C₄-alkyl or —C(═X)R¹⁴,
R¹² and R¹³ furthermore together with the nitrogen atom to which they are attached form a saturated heterocycle which is optionally mono- or polysubstituted by identical or different substituents from the group consisting of halogen and C₁-C₄-alkyl and which has 5 to 8 ring atoms, where the heterocycle may contain 1 or 2 further non-adjacent heteroatoms from the group consisting of oxygen, sulphur and NR⁷,
X represents O (oxygen) or S (sulphur),
R¹⁴ represents hydrogen, C₁-C₄-alkyl, C₁-C₄-alkoxy or —NR¹⁶R¹⁷,
R¹⁵ represents hydrogen, C₁-Cₐ-alkyl, C₁-C₈-alkoxy, C₁-C₄-alkoxy-C₁-C₄-alkyl, C₁-C₄-alkylthio-C₁-C₄-alkyl or C₁-C₆-haloalkyl, where the three radicals R¹⁵ may each be identical or different,
R¹⁶ represents hydrogen or C₁-C₄-alkyl,
R¹⁷ represents hydrogen or C₁-C₄-alkyl,
R¹⁶ and R¹⁷ furthermore together with the nitrogen atom to which they are attached form a saturated heterocycle which is optionally mono- or polysubstituted by identical or different substituents from the group consisting of halogen and C₁-C₄-alkyl and which has 5 to 8 ring atoms, where the heterocycle may contain 1 or 2 further non-adjacent heteroatoms from the group consisting of oxygen, sulphur and NR⁷,
A represents one of the radicals A1 to A18 below 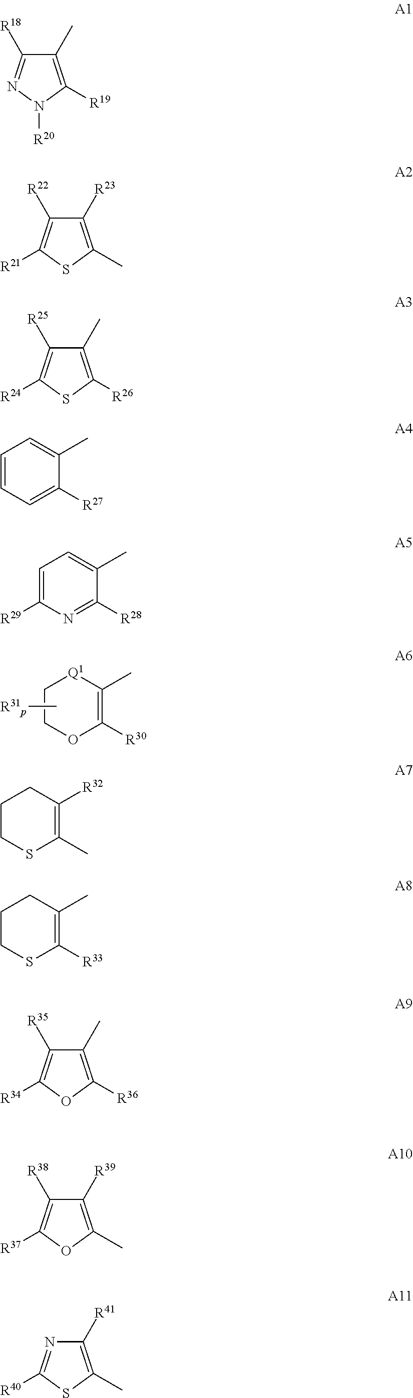 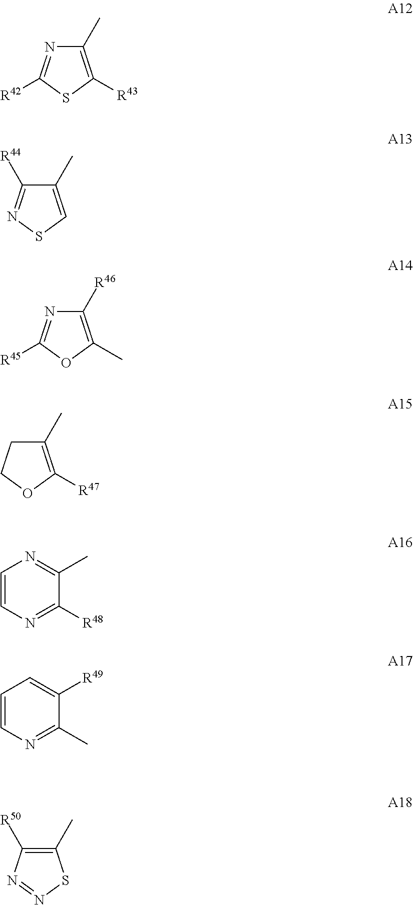
R¹⁸ represents hydrogen, cyano, halogen, nitro, C₁-C₄-alkyl, C₁-C₄-alkoxy, C₁-C₄-alkylthio, C₃-C₆-cycloalkyl, C₁-C₄-haloalkyl, C₁-C₄-haloalkoxy or C₁-C₄-haloalkylthio having in each case 1 to 5 halogen atoms, aminocarbonyl or aminocarbonyl-C₁-C₄-alkyl,
R¹⁹ represents hydrogen, halogen, cyano, C₁-C₄-alkyl, C₁-C₄-alkoxy or C₁-C₄-alkylthio,
R²⁰ represents hydrogen, C₁-C₄-alkyl, hydroxy-C₁-C₄-alkyl, C₂-C₆-alkenyl, C₃-C₆-cycloalkyl, C₁-C₄-alkylthio-C₁-C₄-alkyl, C₁-C₄-alkoxy-C₁-C₄-alkyl, C₁-C₄-haloalkyl, C₁-C₄-haloalkoxy-C₁-C₄-alkyl having in each case 1 to 5 halogen atoms, or phenyl,
R²¹ and R²² independently of one another represent hydrogen, halogen, C₁-C₄-alkyl or C₁-C₄-haloalkyl having 1 to 5 halogen atoms,
R²³ represents halogen, cyano or C₁-C₄-alkyl, or C₁-C₄-haloalkyl or C₁-C₄-haloalkoxy having in each case 1 to 5 halogen atoms,
R²⁴ and R²⁵ independently one another represent hydrogen, halogen, C₁-C₄-alkyl or C₁-C₄-haloalkyl having 1 to 5 halogen atoms,
R26 represents hydrogen, halogen, C₁-C₄-alkyl or C₁-C₄-haloalkyl having 1 to 5 halogen atoms,
R²⁷ represents hydrogen, halogen, hydroxyl, cyano, C₁-C₆-alkyl, C₁-C₄-haloalkyl, C₁-C₄-haloalkoxy or C₁-C₄-haloalkylthio having in each case 1 to 5 halogen atoms,
R²⁸ represents halogen, hydroxyl, cyano, C₁-C₄-alkyl, C₁-C₄-alkoxy, C₁-C₄-alkylthio, C₁-C₄-haloalkyl, C₁-C₄-haloalkylthio or C₁-C₄-haloalkoxy having in each case 1 to 5 halogen atoms,
R²⁹ represents hydrogen, halogen, cyano, C₁-C₄-alkoxy, C₁-C₄-alkylthio, C₁-C₄-haloalkyl, C₁-C₄-haloalkoxy having in each case 1 to 5 halogen atoms, C₁-C₄-alkylsulphinyl or C₁-C₄-alkylsulphonyl,
R³⁰ represents C₁-C₄-alkyl or C₁-C₄-haloalkyl having 1 to 5 halogen atoms,
R³¹ represents C₁-C₄-alkyl,
Q¹ represents S (sulphur), SO, SO₂ or CH₂,
p represents 0, 1 or 2, where R²² represents identical or different radicals if p represents 2,
R³² represents C₁-C₄-alkyl or C₁-C₄-haloalkyl having 1 to 5 halogen atoms,
R³³ represents C₁-C₄-alkyl or C₁-C₄-haloalkyl having 1 to 5 halogen atoms,
R³⁴ and R³⁵ independently of one another represent hydrogen, halogen, amino, C₁-C₄-alkyl or C₁-C₄-haloalkyl having 1 to 5 halogen atoms,
R³⁶ represents hydrogen, halogen, C₁-C₄-alkyl or C₁-C₄-haloalkyl having 1 to 5 halogen atoms,
R³⁷ and R³⁸ independently of one another represent hydrogen, halogen, amino, nitro, C₁-C₄-alkyl or C₁-C₄-haloalkyl having 1 to 5 halogen atoms,
R³⁹ represents hydrogen, halogen, C₁-C₄-alkyl or C₁-C₄-haloalkyl having 1 to 5 halogen atoms,
R⁴⁰ represents hydrogen, halogen, amino, C₁-C₄-alkylamino, di-(C₁-C₄-alkyl)amino, cyano, C₁-C₄-alkyl or C₁-C₄-haloalkyl having 1 to 5 halogen atoms,
R⁴¹ represents halogen, C₁-C₄-alkyl or C₁-C₄-haloalkyl having 1 to 5 halogen atoms,
R⁴² represents hydrogen, halogen, amino, C₁-C₄-alkylamino, di-(C₁-C₄-alkyl)amino, cyano, C₁-C₄-alkyl or C₁-C₄-haloalkyl having 1 to 5 halogen atoms,
R⁴³ represents halogen, C₁-C₄-alkyl or C₁-C₄-haloalkyl having 1 to 5 halogen atoms,
R⁴⁴ represents halogen, C₁-C₄-alkyl or C₁-C₄-haloalkyl having 1 to 5 halogen atoms,
R⁴⁵ represents hydrogen or C₁-C₄-alkyl,
R⁴⁶ represents halogen or C₁-C₄-alkyl,
R⁴⁷ represents C₁-C₄-alkyl or C₁-C₄-haloalkyl having 1 to 5 halogen atoms,
R⁴⁸ represents hydrogen, halogen, C₁-C₄-alkyl or C₁-C₄-haloalkyl having 1 to 5 halogen atoms,
R⁴⁹ represents halogen, hydroxyl, C₁-C₄-alkyl, C₁-C₄alkoxy, C₁-C₄-alkylthio, C₁-C₄haloalkyl, C₁-C₄-haloalkylthio or C₁-C₄-haloalkoxy having in each case 1 to 5 halogen atoms,
R⁵⁰ represents C₁-C₄-alkyl.

2. A thienyl pyridylcarboxamide according to claim 1 of formula

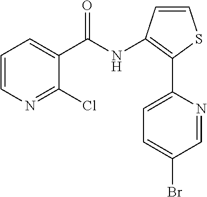

3. A process for preparing thienylpyridylcarboxamides of formula (I) according to claim 1, comprising reacting:
(a) a carbonyl halide of formula (II) 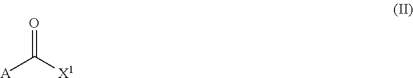
in which
X¹ represents halogen or hydroxyl
with an amine of formula (III) 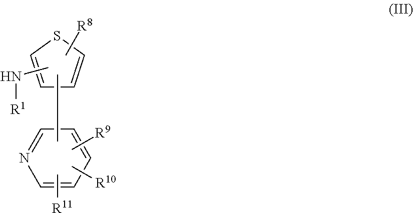
if appropriate in the presence of a coupling agent, if appropriate in the presence of an acid binder and if appropriate in the presence of a diluent,
or
(b) reacting a thienylpyridylcarboxamide of formula (I-a) 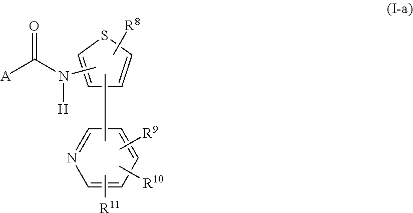
with a halide of formula (IV)
R^{1-B}-Hal (IV)
in which
R^{1-B} represents C₁-C₈-alkyl, C₁-C₆-alkylsulphinyl, C₁-C₆-alkylsulphonyl, C₁-C₄-alkoxy-C₁-C₄-alkyl, C₃-C₈-cycloalkyl; C₁-C₆-haloalkyl, C₁-C₄-haloalkylthio, C₁-C₄-haloalkylsulphinyl, C₁-C₄-haloalkylsulphonyl, halo-C₁-C₄-alkoxy-C₁-C₄-alkyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₁-C₃-alkoxy)carbonyl-C₁-C₃-alkyl having in each case 1 to 13 fluorine, chlorine and/or bromine atoms; (C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl, (C₃-C₈-cycloalkyl)carbonyl; (C₁-C₆-haloalkyl)carbonyl, (C₁-C₆-haloalkoxy)carbonyl, (C₁-C₆-haloalkylthio)carbonyl, (halo-C₁-C₄-alkoxy-C₁-C₄-alkyl)carbonyl, (C₃-C₆-haloalkenyloxy)carbonyl, (C₃-C₆-haloalkynyloxy)carbonyl, (C₃-C₈-halocycloalkyl)carbonyl having in each case 1 to 9 fluorine, chlorine and/or bromine atoms; or —CH₂—C≡C—R^{1-A}, —CH₂—CH═CH—R^{1-A}, —CH═C═CH—R^{1-A}, —C(═O)C(═O)R², —CONR³R⁴ or —CH₂NR⁵R⁶,
R^{1-A}, R², R³, R⁴, R⁵ and R⁶ are as defined in claim 1,
Hal represents chlorine, bromine or iodine
in the presence of a base and in the presence of a diluent.

4. A composition for controlling unwanted microorganisms, comprising at least one thienylpyridylcarboxamide of formula (I) according to claim 1, and an extender and/or surfactant.

5. A thienylpyridylcarboxamide of the formula (I) according to claim 1 that is used to control an unwanted microorganism.

6. A method for controlling an unwanted microorganism, comprising applying a thienylpyridylcarboxamide of formula (I) according to claim 1 to the microorganism and/or a habitat thereof.

7. A process for preparing compositions for controlling an unwanted microorganism, comprising mixing a thienylpyridylcarboxamide of formula (I) according to claim 1 with an extender and/or surfactant.

8. An amine of the formula (III) which is suitable for use and/or an intermediate in making a thienylpyridylcarboxamide of claim 1.

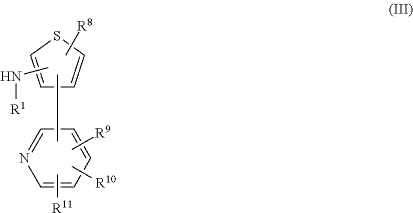

9. A dioxazaborocane derivative of the formula (V) 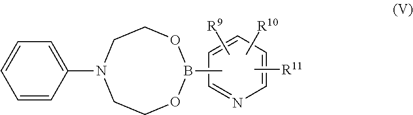
is an intermediate in making a thienylpyridylcarboxamide of claim 1.

10. Carbamates of the formula (VI) 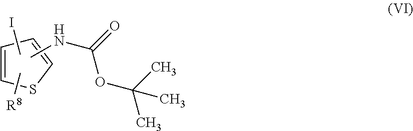
in which R⁸ represents hydrogen, fluorine, chlorine, methyl, isopropyl, methylthio or trifluoromethyl.
